(19) United States
(12) Patent Application Publication
(10) Pub. No.: US 2012/0258957 A1
(43) Pub. Date: Oct. 11, 2012

(54) HETEROCYCLIC DERIVATIVES

(76) Inventors: Matilda Jane Bingham, Newhouse (GB); Andrew Dunbar, Newhouse (GB); Margaret Jean Laats, Newhouse (GB); Grant Wishart, Newhouse (GB)

(21) Appl. No.: 13/511,238

(22) PCT Filed: Nov. 22, 2010
(86) PCT No.: PCT/EP2010/067904

§ 371 Date: Jun. 25, 2012

Related U.S. Application Data
(60) Provisional application No. 61/263,639, filed on Nov. 23, 2009.

(30) Foreign Application Priority Data
Nov. 23, 2009 (EP) 09176778.0

(51) Int. Cl. A61K 31/4725; A61P 25/18; A61P 3/04; C07D 401/14; C07D 401/06; C07D 471/04; A61K 31/55; A61P 25/00; A61P 3/10

(52) U.S. Cl. 514/217.01; 546/140; 514/308; 546/122; 514/300; 540/594; 546/146; 514/307

(57) ABSTRACT

The present invention relates to a heterocyclic derivative according to formula I

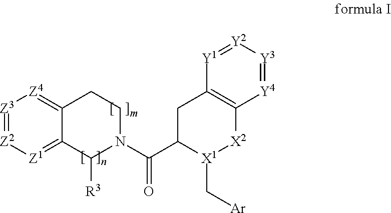

wherein the variables are defined as in the specification, or to a pharmaceutically acceptable salt or solvate thereof. The present invention also relates to a pharmaceutical composition comprising said heterocyclic derivatives and to their use in therapy, for instance in the treatment or prevention of disorders or diseases influenced by modulation of orexins, such as sleep disorders.

The present invention relates to heterocyclic derivatives, to pharmaceutical compositions comprising these compounds and to their use in therapy, in particular to their use in the treatment or prevention of neurological and psychiatric disorders and diseases in which the orexin receptors are involved.

The neuropeptides known as orexin-A and -B (also known as hypocretin-1 and hypocretin-2) are 33- and 28-residue peptides, respectively. The sequence of orexin-A is conserved in several mammalian species, whereas, mouse and rat orexin-B peptides are identical, but human orexin-B has two amino acid substitutions compared with the rodent sequences, P2S and N18S (rodents to human). Orexin-A and -B bind and activate two closely related G protein coupled receptors, the orexin-1 (OX₁R) and orexin-2 (OX₂R) receptors, to evoke a variety of biological effects. Orexin-A has high affinity for both receptors while orexin-B has higher affinity for OX₂R. OX₁R and OX₂R are both highly conserved across mammalian species with sequence homology of 91-98% between human, pig, dog, mouse and rat (de Lecea L, PNAS, 1998, 95:322-327).

Anatomical studies show that neurons synthesising orexin project to brain monoaminergic cell groups including cells containing norepinephrine, serotonin, histamine and dopamine (Peyron C et al, The Journal of Neuroscience, 1998, 18:9996-10015). Orexin cells also project strongly to cholinergic cells in the basal forebrain and the brainstem (Peyron C et al, The Journal of Neuroscience, 1998, 18:9996-10015; Sakurai T, et al, Brain Research 1999, 827:243-260.8) and receive projections from the medial and ventrolateral preoptic area, medial bed nucleus of the stria terminalis, lateral septum, posterior hypothalamus, ventral tegmental area (VTA), locus coeruleus and dorsal raphe (Scammell T E, et al, Neurology, 2005, 494(5):845-861). The distribution of orexin-containing neurons in the hypothalamus and associated circuitry supports the diverse range of biological actions attributed to this family of neuropeptides including a role in feeding and energy homeostasis, the sleep-wake cycle, neuroendocrine homeostasis, nociceptive processing, cardiovascular functions, gastric acid secretion, reward systems, psychiatric and neurological disease for example Parkinson's disease and Alzheimer's disease (Cai, J et al, Current Opinion in Drug Discovery, 2006, 9(5):551-559; Kenji, N. et al, Journal of the Neurological Sciences 2006, 250(1-2):120-123; Holtzman D. M. et al, Science, 24 Sep. 2009 [DOI: 10.1126/science.1180962] (in Science Express Reports)).

Several non-peptide low-molecular weight antagonists are known which are selective for the orexin receptors (for a recent review see Cai J, et al, Expert Opinion on Therapeutic Patents 2006, 16(5): 631-646.) and certain orexin antagonists are disclosed in PCT patent applications; WO 2004041816, WO 2002051838, WO2002051232, WO200185693, WO200168609, WO2004085403, WO 2008122513. In spite of the availability of these compounds, however, there exists a need for further non-peptide orexin selective antagonists which are both safe and effective.

In a first aspect, the present invention provides a heterocyclic derivative having the formula I

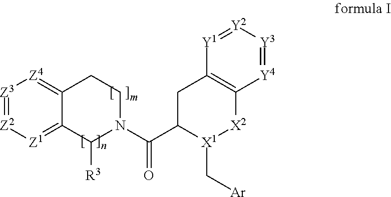

wherein
X¹ and X² are independently CH or N with the proviso that one of X¹ and X² is N;

### Y¹-Y⁴ are CR¹ or 1-2 of Y¹ to Y⁴ are N;

### Z¹-Z⁴ are CR² or 1-2 of Z¹ to Z⁴ are N;

Each R¹ and R² is independently H, C₁₋₆alkyl, C₂₋₆alkenyl, C₂₋₆alkynyl, C₁₋₆alkoxy, C₂₋₆alkenyloxy, C₃₋₇cycloalkyl, C₃₋₇cycloalkyloxy, OH, halogen or CN said C₁₋₆alkyl and C₁₋₆alkoxy being optionally substituted with one or more halogens;
Each R³ is independently H, C₁₋₆alkyl or C₃₋₇cycloalkyl said C₁₋₆alkyl and C₃₋₇cycloalkyl being optionally substituted with one or more substituent independently selected from halogen, OH, CN, NR⁴R⁵ and C₁₋₆alkoxy;
R⁴ and R⁵ are independently H or C₁₋₆alkyl or R⁴ and R⁵ together with the N to which they are bonded form a 4-7 membered heterocyclic ring;
Ar is C₆₋₁₀aryl or a 5-10 membered heteroaryl ring system comprising 1-3 heteroatoms independently selected from N, O and S, said C₆₋₁₀aryl or 5-10 membered heteroaryl being optionally substituted with 1-3 R⁶;
Each R⁶ is independently selected from C₁₋₆alkyl, C₂₋₆alkenyl, C₂₋₆alkynyl, C₁₋₆alkoxy, C₂₋₆alkenyloxy, C₃₋₇cycloalkyl, C₃₋₇cycloalkyloxy, OH, halogen, CN and a 5-6 membered heteroaryl comprising 1-2 heteroatoms selected from N, O and S, said C₁₋₆alkyl and C₁₋₆alkoxy being optionally substituted with one or more halogens;
m is 0 or 1 and
n is 1 or 2
or a pharmaceutically acceptable salt or solvate thereof.

The term C₁₋₆alkyl, as used herein, represents a branched or unbranched alkyl group having 1-6 carbon atoms. Examples of such groups are methyl, ethyl, isopropyl, tertiary-butyl, pentyl and hexyl.

The term C₂₋₆alkenyl, as used herein, represents a branched or unbranched alkenyl group having 2-6 carbon atoms and at least one double bond. Examples of such groups are ethenyl and isopropenyl.

The term C₂₋₆alkynyl, as used herein, represents a branched or unbranched alkynyl group having 2-6 carbon atoms and at least one triple bond. Examples of such groups are ethynyl and isopropynyl.

The term C₃₋₇cycloalkyl, as used herein, represents a branched or unbranched cyclic alkyl group having 3-7 carbon atoms. Examples of such groups are cyclopropyl, cyclopentyl and 2-methylcyclopentyl.

The term C₁₋₆alkyloxy, as used herein, represents a branched or unbranched alkyloxy group having 1-6 carbon atoms. Examples of such groups are methoxy, ethoxy, isopropyloxy and tertiary-butyloxy.

The term C₂₋₆alkenyloxy, as used herein, represents a branched or unbranched alkenyloxy group having 2-6 carbon atoms. Examples of such groups are ethenyloxy and isopropenyloxy.

The term C₃₋₇cycloalkyloxy, as used herein, represents a branched or unbranched cyclic alkyloxy group having 3-7 carbon atoms. Examples of such groups are cyclopropyloxy, cyclopentyloxy and 2-methylcyclopentyloxy.

The term C₆₋₁₀aryl, as used herein, represents an aromatic group having 6-10 carbon atoms and comprising at least one aromatic ring. Examples of such groups include phenyl and naphthyl.

The term halogen, as used herein, represents a fluorine, chlorine, bromine or iodine.

The term 5-10 membered heteroaryl ring system comprising 1-3 heteroatoms selected from N, O and S, as used herein, represents a monocyclic or fused bicyclic 5-10 membered heteroaryl ring system comprising 1-3 heteroatoms selected from N, O and S. Examples of such groups include furanyl, thienyl, oxazolyl, pyrazolyl, imidazolyl, pyrrolyl, pyridinyl, pyrimidyl, indolyl, benzthienyl, benzthiazolyl and quinolinyl. Similarly, examples of 5-6 membered heteroaryl comprising 1-2 heteroatoms selected from N, O and S include, furanyl, thienyl, oxazolyl, pyrazolyl, imidazolyl, pyrrolyl, pyridinyl and pyrimidyl.

Examples of 4-7 membered heterocyclic rings formed by R⁴ and R⁵ together with the N to which they are bonded include piperidine and pyrrolidine.

In one embodiment of the present invention, X¹ is N and X² is CH.

In another embodiment of the present invention, X¹ is CH and X² is N.

In another embodiment of the present invention, Y¹-Y⁴ are CR¹, wherein each R¹ is selected independently and has the previously defined meanings. In another embodiment, Y¹-Y⁴ are CR¹, wherein each R¹ is independently H or methyl. In a further embodiment, Y¹-Y⁴ are CH. In a further embodiment, one of Y¹-Y⁴ is N and the others are CH.

In another embodiment of the present invention, Z¹-Z⁴ are CR², wherein each R² is selected independently and has the previously defined meanings. In another embodiment, one or two of Z¹-Z⁴ are C(OCH₃) and the others are CH. In another embodiment, Z¹ and Z⁴ are CH and Z² and Z³ are C(OCH₃). In a further embodiment, one of Z¹-Z⁴ is N and the others are CH or C(OCH₃).

In another embodiment of the present invention, R³ is H or C₁₋₄alkyl, optionally substituted with CN or OH. In a further embodiment, R³ is C₃₋₅cycloalkyl, optionally substituted with CN or OH. In a further embodiment, R³ is H, methyl, ethyl, isopropyl or cyclopropyl, said methyl, ethyl, isopropyl and cyclopropyl being optionally substituted with CN or OH. In a further embodiment, R³ is H, methyl or ethyl, said methyl and ethyl being optionally substituted with CN or OH. In a further embodiment, R³ is H, methyl, CH₂OH, or CH₂CN.

In a further embodiment, Ar is phenyl optionally substituted with 1-3 R⁶, wherein each R⁶ is selected independently and has the previously defined meanings. In a further embodiment, Ar is phenyl optionally substituted with 1-3 substituents selected from fluoro, chloro, methyl, trifluoromethyl, methoxyl, trifluoromethoxyl, ethoxyl, and CN.

In a further embodiment, Ar is pyridyl, indole or pyrazole, said pyridyl, indole and pyrazole being optionally substituted with 1-3 substituents selected from fluoro, chloro, methyl, trifluoromethyl, methoxyl, trifluoromethoxyl, ethoxyl, and CN.

In a further embodiment, Ar is pyridyl optionally substituted with 1-3 substituents selected from fluoro, chloro, methyl, trifluoromethyl, methoxyl, trifluoromethoxyl, ethoxyl, and CN.

In a further embodiment of the present invention m is 1.

In a further embodiment of the present invention n is 1.

A further embodiment of the present invention is a compound of the formula Ia

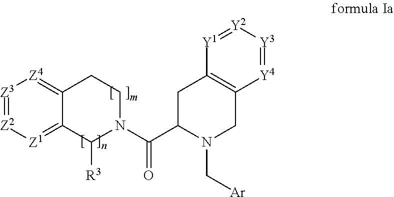

wherein R¹, R² n, m, Z¹, Z², Z³, Z⁴, Y¹, Y², Y³, Y⁴ and Ar have the meanings previously defined and R³ is H, CH₃, CH₂OH, or CH₂CN; or a pharmaceutically acceptable salt thereof.

A further embodiment of the present invention is a compound of the formula Ib

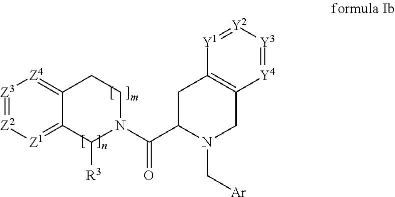

wherein, Y¹-Y⁴ are CR¹, wherein each R¹ is independently H or methyl, R³ is H, CH₃, CH₂OH, or CH₂CN and wherein Z¹-Z⁴, R², n, m, and Ar have the meanings previously defined; or a pharmaceutically acceptable salt thereof.

A further embodiment of the present invention is a compound of the formula Ic

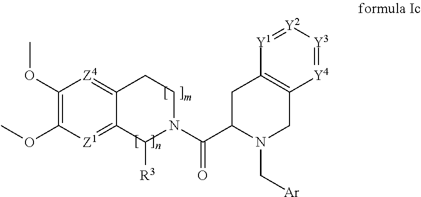

wherein R¹, n, m, Y¹, Y², Y³, Y⁴ Z¹, Z⁴ and Ar have the meanings previously defined and R³ is H, CH₃, CH₂OH, or CH₂CN; or a pharmaceutically acceptable salt thereof.

A further embodiment of the present invention is a compound of the formula Id

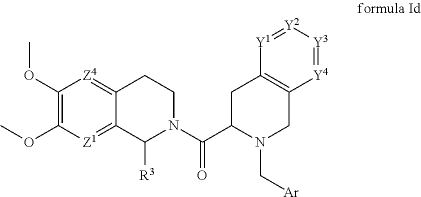

wherein Y¹, Y², Y³, Y⁴, Z¹, Z⁴ and Ar have the meanings previously defined and R³ is H, CH₃, CH₂OH, or CH₂CN; or a pharmaceutically acceptable salt thereof.

A further embodiment of the present invention is a compound of the formula Ie

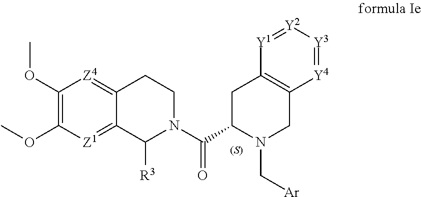

wherein Y¹, Y², Y³, Y⁴, Z¹, Z⁴ and Ar have the meanings previously defined and R³ is H, CH₃, CH₂OH, or CH₂CN; or a pharmaceutically acceptable salt thereof.

In a further embodiment is a heterocyclic derivative selected from:
(S)-(6,7-dimethoxy-3,4-dihydroisoquinolin-2(1H)-yl)(2-(2-(trifluoromethoxy)benzyl)-1,2,3,4-tetrahydroisoquinolin-3-yl)methanone;
(S)-(1-ethyl-6,7-dimethoxy-3,4-dihydroisoquinolin-2(1H)-yl)(2-(2-methoxybenzyl)-1,2,3,4-tetrahydroisoquinolin-3-yl)methanone;
(S)-(1-cyclopropyl-6,7-dimethoxy-3,4-dihydroisoquinolin-2(1H)-yl)(2-(2-methoxybenzyl)-1,2,3,4-tetrahydroisoquinolin-3-yl)methanone;
(S)-(2-benzyl-1,2,3,4-tetrahydroisoquinolin-3-yl)(6,7-dimethoxy-3,4-dihydroisoquinolin-2(1H)-yl)methanone;
(S)-(2-benzyl-1,2,3,4-tetrahydroisoquinolin-3-yl)(6,7-dimethoxy-1-methyl-3,4-dihydroisoquinolin-2(1H)-yl)methanone;
(S)-(2-(2-chlorobenzyl)-1,2,3,4-tetrahydroisoquinolin-3-yl)(6,7-dimethoxy-3,4-dihydroisoquinolin-2(1H)-yl)methanone;
(S)-(6,7-dimethoxy-3,4-dihydroisoquinolin-2(1H)-yl)(2-(2-(trifluoromethyl)benzyl)-1,2,3,4-tetrahydroisoquinolin-3-yl)methanone;
(S)-(6,7-dimethoxy-3,4-dihydroisoquinolin-2(1H)-yl)(2-(2-fluorobenzyl)-1,2,3,4-tetrahydroisoquinolin-3-yl)methanone;
(S)-(6,7-dimethoxy-1-methyl-3,4-dihydroisoquinolin-2(1H)-yl)(2-(2-methoxybenzyl)-1,2,3,4-tetrahydroisoquinolin-3-yl)methanone;
((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride;
((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride;
(S)-2-(6,7-dimethoxy-2-(2-(2-methoxybenzyl)-1,2,3,4-tetrahydroisoquinoline-3-carbonyl)-1,2,3,4-tetrahydroisoquinolin-1-yl)acetonitrile;
(S)-2-(2-(2-(2-fluorobenzyl)-1,2,3,4-tetrahydroisoquinoline-3-carbonyl)-6,7-dimethoxy-1,2,3,4-tetrahydroisoquinolin-1-yl)acetonitrile;
((R)-6,7-dimethoxy-1-methyl-3,4-dihydroisoquinolin-2(1H)-yl)((S)-2-(2-fluorobenzyl)-1,2,3,4-tetrahydroisoquinolin-3-yl)methanone hydrochloride;
(S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 2);
(S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 1);
(S)-(6,7-dimethoxy-1-methyl-3,4-dihydroisoquinolin-2(1H)-yl)(2-(2-fluoro-6-methoxybenzyl)-1,2,3,4-tetrahydroisoquinolin-3-yl)methanone;
((R)-6,7-dimethoxy-1-methyl-3,4-dihydroisoquinolin-2(1H)-yl)((S)-2-((4-methoxypyridin-3-yl)methyl)-1,2,3,4-tetrahydroisoquinolin-3-yl)methanone;
((R)-6,7-dimethoxy-1-methyl-3,4-dihydroisoquinolin-2(1H)-yl)((S)-2-((4-(trifluoromethyl)pyridin-3-yl)methyl)-1,2,3,4-tetrahydroisoquinolin-3-yl)methanone;
((S)-2-((1H-indol-4-yl)methyl)-1,2,3,4-tetrahydroisoquinolin-3-yl)((R)-6,7-dimethoxy-1-methyl-3,4-dihydroisoquinolin-2(1H)-yl)methanone;
((S)-2-((3-chloropyridin-4-yl)methyl)-1,2,3,4-tetrahydroisoquinolin-3-yl)((R)-6,7-dimethoxy-1-methyl-3,4-dihydroisoquinolin-2(1H)-yl)methanone;
((R)-6,7-dimethoxy-1-methyl-3,4-dihydroisoquinolin-2(1H)-yl)((S)-2-((3-(trifluoromethyl)pyridin-2-yl)methyl)-1,2,3,4-tetrahydroisoquinolin-3-yl)methanone;
((S)-2-((4-chloro-1-methyl-1H-pyrazol-3-yl)methyl)-1,2,3,4-tetrahydroisoquinolin-3-yl)((R)-6,7-dimethoxy-1-methyl-3,4-dihydroisoquinolin-2(1H)-yl)methanone;
((R)-6,7-dimethoxy-1-methyl-3,4-dihydroisoquinolin-2(1H)-yl)((S)-2-((3-fluoropyridin-2-yl)methyl)-1,2,3,4-tetrahydroisoquinolin-3-yl)methanone;
(S)-(6,7-dimethoxy-3,4-dihydroisoquinolin-2(1H)-yl)(2-(2-methoxybenzyl)-1,2,3,4-tetrahydroisoquinolin-3-yl)methanone hydrochloride;
(S)-(2-(2,3-dichlorobenzyl)-1,2,3,4-tetrahydroisoquinolin-3-yl)(6,7-dimethoxy-3,4-dihydroisoquinolin-2(1H)-yl)methanone hydrochloride and
(S)-(2-(2-fluorobenzyl)-1,2,3,4-tetrahydroisoquinolin-3-yl)(1-(hydroxymethyl)-7-isopropoxy-6-methoxy-3,4-dihydroisoquinolin-2(1H)-yl)methanone;
   or a pharmaceutically acceptable salt or solvate thereof.

The compounds of the present invention are prepared by methods well known in the art of organic chemistry, see for example, J. March, ‘Advanced Organic Chemistry’ 4^{th} Edition, John Wiley and Sons. During synthetic sequences it may be necessary and/or desirable to protect sensitive or reactive groups on any of the molecules concerned. This is achieved by means of conventional protecting groups, such as those described in T. W. Greene and P.G.M. Wutts ‘Protective Groups in Organic Synthesis’ 3^{rd} Edition, John Wiley and Sons, 1999. The protective groups are optionally removed at a convenient subsequent stage using methods well known in the art. Several methods for preparing compounds of this invention are illustrated in the following schemes and examples. Starting materials are made according to procedures known in the art or as illustrated herein. The compounds of the present invention can be prepared in a variety of fashions.

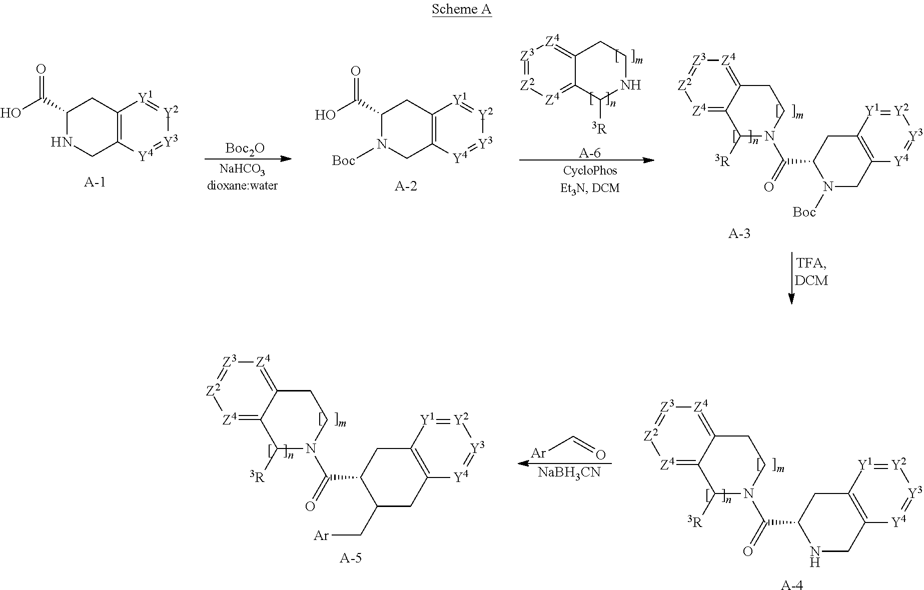

Compounds of formula A-5, wherein R¹, R², R³, n, m, Z¹, Z², Z³, Z⁴, Y¹, Y², Y³, Y⁴ and Ar have the meanings as previously defined, can be prepared according to the route outlined in Scheme A, from compounds of formula A-1 by Boc protection using Boc₂O in the present of a base such as sodium bicarbonate, in a suitable solvent such as dioxane:water. Coupling of acid A-2 with amine A-6 can be performed using a variety of coupling agents known to those skilled in the art, for example Cyclophos, in the presence of a base such as Et₃N. The resultant intermediate A-3 can be deprotected by an acid, for example TFA, in a suitable solvent such as DCM to afford amine A-4. Amine A-4 can undergo reductive amination with an aromatic or heteroaromatic aldehyde in the presence of a reducing agent such as sodium cyanoborohydride to afford derivatives of formula A-5 (Scheme A).

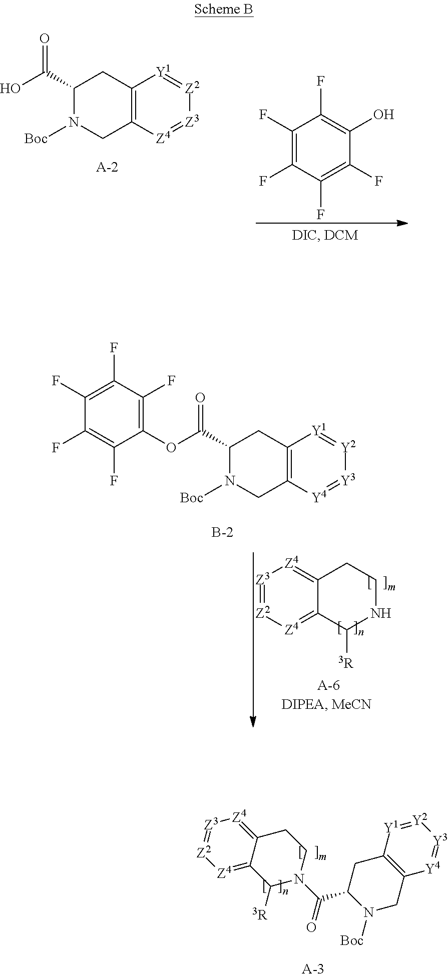

Compounds of formula A-3, wherein R¹, R², R³, n, m, Z¹, Z², Z³, Z⁴, Y¹, Y², Y³, Y⁴ and Ar have the meanings as previously defined, may also be prepared from compounds such as A-2 by coupling with pentafluorophenol using a suitable coupling agent such as DIC. Followed by reaction with an amine of formula A-6 (Scheme B)

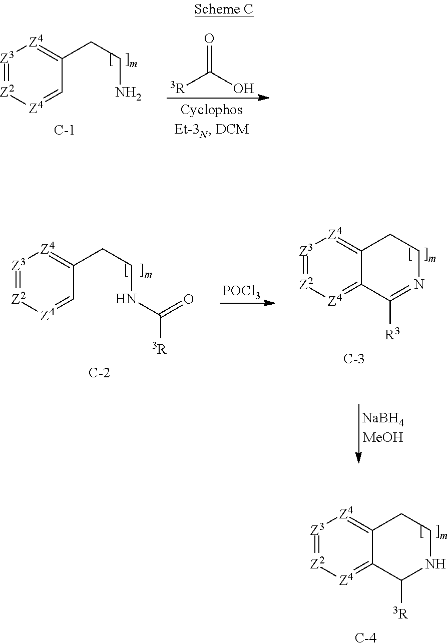

Compounds of formula C-4, wherein R¹, R², R³ m, Z¹, Z₂, Z³, and Z⁴ have the meanings as previously defined and n is 1 can be prepared from compounds of formula C-1, by coupling with a carboxylic acid using a variety of coupling agents known to those skilled in the art, for example Cyclophos, in the presence of a base such as Et₃N to afford amide C-2. Amide C-2 can be cyclised under dehydrating conditions with a reagent such as phosphorus oxychloride to afford imines of formula C-3 which can be reduced with a suitable reducing agent such as NaBH₄ in an appropriate solvent such as methanol to afford amines of formula C-4(Scheme C).

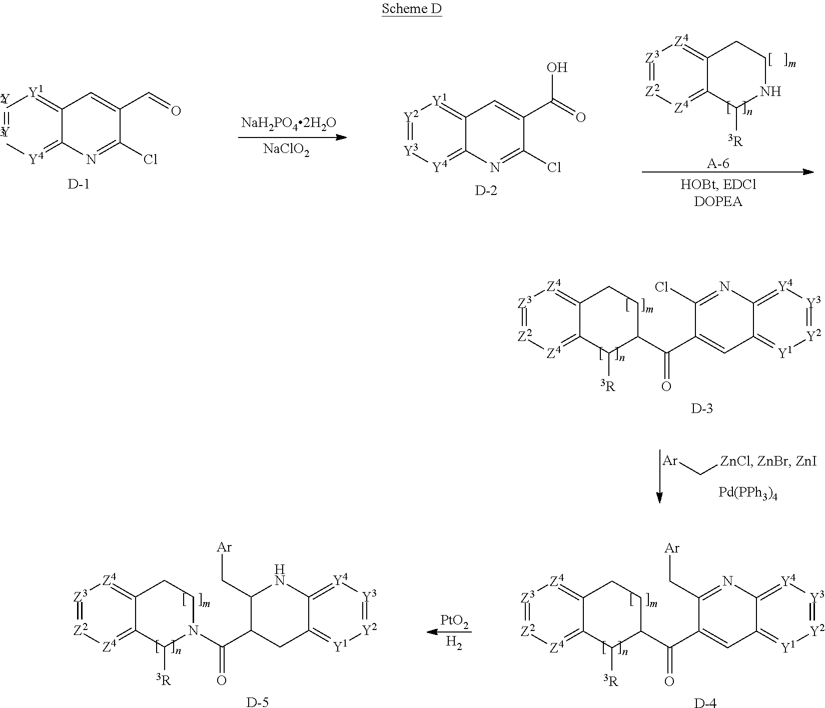

Compounds of formula D-5, wherein R¹, R², R³ n, m, Z¹, Z², Z³, Z⁴, Y¹, Y², Y³, Y⁴ and Ar have the meanings previously defined, may be synthesised from compounds of formula D-2, obtained by oxidation of a compounds of formula D-1, followed by coupling with an amine such as A-6 using a variety of coupling agents known to those skilled in the art, for example HOBt and EDCl in the presence of a suitable base such as DIPEA, then metal catalysed coupling with a suitable benzylic organometallic such as an benzylzinc halide and a catalyst such as Pd(PPh₃)₄ followed by reduction with hydrogen gas in the presence of a suitable catalyst such as platinum(IV) oxide hydrate.

It will be readily appreciated by one skilled in the art that the compounds of general formula I can be prepared using the general procedures and/or reaction sequences described above in any suitable order.

The present invention also includes within its scope all stereoisomeric forms of the heterocyclic derivatives resulting, for example, because of configurational or geometrical isomerism. Such stereoisomeric forms are enantiomers, diastereoisomers, cis and trans isomers etc. In the case of the individual enantiomers of compounds of formula I or salts or solvates thereof, the present invention includes the aforementioned stereoisomers substantially free, i.e., associated with less than 5%, preferably less than 2% and in particular less than 1% of the other enantiomer. Mixtures of stereoisomers in any proportion, for example a racemic mixture comprising substantially equal amounts of two enantiomers are also included within the scope of the present invention.

For chiral compounds, methods for asymmetric synthesis whereby the pure stereoisomers are obtained are well known in the art, e.g., synthesis with chiral induction, synthesis starting from chiral intermediates, enantioselective enzymatic conversions, separation of stereoisomers using chromatography on chiral media. Such methods are described in Chirality In Industry (edited by A. N. Collins, G. N. Sheldrake and J. Crosby, 1992; John Wiley). Likewise methods for synthesis of geometrical isomers are also well known in the art.

The heterocyclic derivatives of the present invention, in the form as a free base, are isolated from reaction mixtures as pharmaceutically acceptable salts. These salts are also obtained by treatment of said free base with an organic or inorganic acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, citric acid, benzoic acid and ascorbic acid.

The heterocyclic derivatives of the present invention also exist as amorphous forms. Multiple crystalline forms are also possible. All these physical forms are included within the scope of the present invention.

Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods.

Analytical techniques such as, for example I. R. spectroscopy, show the presence of the solvent (or water) in the crystals as a solvate (or hydrate).

The present invention also embraces isotopically-labelled compounds of the compounds described and claimed herein which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I can generally be prepared by following procedures analogous to those disclosed in the Schemes and/or in the Examples hereinbelow, by substituting an appropriate isotopically labelled reagent for a non-isotopically labelled reagent.

Prodrugs of the compounds of the invention are also contemplated within the scope of the invention. A prodrug is a compound which acts as a drug precursor which, upon administration to a subject, undergoes conversion by metabolic or other chemical processes to yield a heterocyclic derivative of formula I or a solvate or salt thereof. For example, where R³ is hydroxymethyl the hydroxyl group may be capped as, for example, an ester or a carbamate, which upon administration to a subject will undergo conversion back to the free hydroxyl group. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In a further aspect, the heterocyclic derivatives of the present invention and their pharmaceutically acceptable salts and solvates are useful in therapy. Accordingly, the heterocyclic derivatives of the present invention have utility in treating, preventing, ameliorating, controlling or reducing the risk of a variety of neurological and psychiatric disorders associated with orexin receptors, including one or more of the following conditions or diseases: sleep disorders, sleep disturbances, including enhancing sleep quality, improving sleep quality, increasing sleep efficiency, augmenting sleep maintenance; increasing the value which is calculated from the time that a subject sleeps divided by the time that a subject is attempting to sleep; improving sleep initiation; decreasing sleep latency or onset (the time it takes to fall asleep); decreasing difficulties in falling asleep; increasing sleep continuity; decreasing the number of awakenings during sleep; decreasing intermittent wakings during sleep; decreasing nocturnal arousals; decreasing the time spent awake following the initial onset of sleep; increasing the total amount of sleep; reducing the fragmentation of sleep; altering the timing, frequency or duration of REM sleep bouts; altering the timing, frequency or duration of slow wave (i.e. stages 3 or 4) sleep bouts; increasing the amount and percentage of stage 2 sleep; promoting slow wave sleep; enhancing EEG-delta activity during sleep; decreasing nocturnal arousals, especially early morning awakenings; increasing daytime alertness; reducing daytime drowsiness; treating or reducing excessive daytime sleepiness; increasing satisfaction with the intensity of sleep; increasing sleep maintenance; idiopathic insomnia; sleep problems; insomnia, hypersomnia, idiopathic ‘hypersomnia, repeatability hypersomnia, intrinsic hypersomnia, narcolepsy, interrupted sleep, sleep apnea, wakefulness, nocturnal myoclonus, REM sleep interruptions, jet-lag, shift workers’ sleep disturbances, dyssomnias, night terror, insomnias associated with depression, emotional/mood disorders, Alzheimer's disease or cognitive impairment, as well as sleep walking and enuresis, and sleep disorders which accompany aging; Alzheimer's sundowning; conditions associated with circadian rhythmicity as well as mental and physical disorders associated with travel across time zones and with rotating shift-work schedules, conditions due to drugs which cause reductions in REM sleep as a side effect; fibromyalgia; syndromes which are manifested by non-restorative sleep and muscle pain or sleep apnea which is associated with respiratory disturbances during sleep; conditions which result from a diminished quality of sleep; increasing learning; augmenting memory; increasing retention of memory; eating disorders associated with excessive food intake and complications associated therewith, compulsive eating disorders, obesity (due to any cause, whether genetic or environmental), obesity-related disorders including overeating and bulimia nervosa, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia, metabolic syndrome, also known as syndrome X, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, increased anesthetic risk, reducing the risk of secondary outcomes of obesity, such as reducing the risk of left ventricular hypertrophy; diseases or disorders where abnormal oscillatory activity occurs in the brain, including depression, migraine, neuropathic pain, Parkinson's disease, psychosis and schizophrenia, as well as diseases or disorders where there is abnormal coupling of activity, particularly through the thalamus; enhancing cognitive function; enhancing memory; increasing memory retention; increasing immune response; increasing immune function; hot flashes; night sweats; extending life span; schizophrenia; muscle-related disorders that are controlled by the excitation/relaxation rhythms imposed by the neural system such as cardiac rhythm and other disorders of the cardiovascular system; conditions related to proliferation of cells such as vasodilation or vasorestriction and blood pressure; cancer; cardiac arrhythmia; hypertension; congestive heart failure; conditions of the genital/urinary system; disorders of sexual function and fertility; adequacy of renal function; responsivity to anesthetics; mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder, mood disorders due to a general medical condition, and substance-induced mood disorders; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; acute neurological and psychiatric disorders such as cerebral deficits subsequent to cardiac bypass surgery and grafting, stroke, ischemic stroke, cerebral ischemia, spinal cord trauma, head trauma, perinatal hypoxia, cardiac arrest, hypoglycemic neuronal damage; Huntington's Chorea; amyotrophic lateral sclerosis; multiple sclerosis; ocular damage; retinopathy; cognitive disorders; idiopathic and drug-induced Parkinson's disease; muscular spasms and disorders associated with muscular spasticity including tremors, epilepsy, convulsions; cognitive disorders including dementia (associated with Alzheimer's disease, ischemia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced psychotic disorder; substance-related disorders and addictive behaviors (including substance-induced delirium, persisting dementia, persisting amnestic disorder, psychotic disorder or anxiety disorder; tolerance, addictive feeding, dependence or withdrawal from substances including alcohol, amphetamines, cannabis, cocaine, hallucinogens, inhalants, nicotine, opioids, phencyclidine, sedatives, hypnotics or anxiolytics); movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), chronic fatigue syndrome, fatigue, including Parkinson's fatigue, multiple sclerosis fatigue, fatigue caused by a sleep disorder or a circadian rhythm disorder, medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, and dyskinesias [including tremor (such as rest tremor, essential tremor, postural tremor and intention tremor)], chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics, and symptomatic tics), restless leg syndrome and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia); attention deficit/hyperactivity disorder (ADHD); conduct disorder; migraine (including migraine headache); urinary incontinence; substance tolerance, substance withdrawal (including, substances such as opiates, nicotine, tobacco products, alcohol, benzodiazepines, cocaine, sedatives, hypnotics, etc.); psychosis; schizophrenia; anxiety (including generalized anxiety disorder, panic disorder, and obsessive compulsive disorder); mood disorders (including depression, mania, bipolar disorders); trigeminal neuralgia; hearing loss; tinnitus; neuronal damage including ocular damage; retinopathy; macular degeneration of the eye; emesis; brain edema; pain, including acute and chronic pain states, severe pain, intractable pain, inflammatory pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain, post-traumatic pain, trigeminal neuralgia, migraine and migraine headache.

The present invention further includes a method for the treatment of a mammal, including a human, suffering from or liable to suffer from any of the aforementioned disorders, which comprises administering an effective amount of a heterocyclic derivative of the present invention or a pharmaceutically acceptable salt or solvate thereof. By effective amount or therapeutically effective amount is meant an amount of compound or a composition of the present invention effective in inhibiting the above-noted diseases and thus producing the desired therapeutic, ameliorative, inhibitory or preventative effect.

Thus, in specific embodiments the present invention provides methods for: enhancing the quality of sleep; augmenting sleep maintenance; increasing REM sleep; increasing stage 2 sleep; decreasing fragmentation of sleep patterns; treating insomnia; enhancing cognition; increasing memory retention; treating or controlling obesity; treating or controlling depression; treating, controlling, ameliorating or reducing the risk of epilepsy, including absence epilepsy; treating or controlling pain, including neuropathic pain; treating or controlling Parkinson's disease; treating or controlling Alzheimer's disease; treating or controlling psychosis; or treating, controlling, ameliorating or reducing the risk of schizophrenia, in a mammalian patient in need thereof which comprises administering to the patient a therapeutically effective amount of a compound of the present invention.

The amount of a heterocyclic derivative of the present invention or a pharmaceutically acceptable salt or solvate thereof, also referred to herein as the active ingredient, which is required to achieve a therapeutic effect will, of course, vary with the particular compound, the route of administration, the age and condition of the recipient, and the particular disorder or disease being treated.

A suitable daily dose for any of the above mentioned disorders will be in the range of 0.001 to 50 mg per kilogram body weight of the recipient (e.g. a human) per day, preferably in the range of 0.01 to 20 mg per kilogram body weight per day. The desired dose may be presented as multiple sub-doses administered at appropriate intervals throughout the day.

Whilst it is possible for the active ingredient to be administered alone, it is preferable to present it as a pharmaceutical formulation. The present invention therefore also provides a pharmaceutical composition comprising a heterocyclic derivative according to the present invention in admixture with one or more pharmaceutically acceptable auxiliaries, such as the ones described in Gennaro et. al., Remmington: The Science and Practice of Pharmacy, 20^{th} Edition, Lippincott, Williams and Wilkins, 2000; see especially part 5: pharmaceutical manufacturing. Suitable auxiliaries are described e.g., in the Handbook of Pharmaceutical Excipients, 2^{nd} Edition; Editors A. Wade and P. J. Weller, American Pharmaceutical Association, Washington, The Pharmaceutical Press, London, 1994. Compositions include those suitable for oral, nasal, topical (including buccal, sublingual and transdermal), parenteral (including subcutaneous, intravenous and intramuscular) or rectal administration.

The mixtures of a heterocyclic derivative according to the present invention and one or more pharmaceutically acceptable auxiliary or auxiliaries may be compressed into solid dosage units, such as tablets, or be processed into capsules or suppositories. By means of pharmaceutically suitable liquids the heterocyclic derivatives can also be applied as an injection preparation in the form of a solution, suspension, emulsion, or as a spray, e.g., a nasal or buccal spray. For making dosage units e.g., tablets, the use of conventional additives such as fillers, colorants, polymeric binders and the like is contemplated. In general, any pharmaceutically acceptable additive can be used. The heterocyclic derivatives of the invention are also suitable for use in an implant, a patch, a gel or any other preparation for immediate and/or sustained release.

Suitable fillers with which the pharmaceutical compositions can be prepared and administered include lactose, starch, cellulose and derivatives thereof, and the like, or mixtures thereof used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The present invention further includes a pharmaceutical composition, as hereinbefore described, in combination with packaging material suitable for said composition, said packaging material including instructions for the use of the composition for the use as hereinbefore described.

The following Examples further illustrate the compounds of the present invention and methods for their synthesis. The following examples are put forth so as to provide those of ordinary skill in the art with disclosure and description of how compounds, compositions and methods herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were used without further purification.

### Methods

General Chemical Procedures. All reagents were either purchased from common commercial sources or synthesised according to literature procedures using commercially available starting materials. Mass spectra were recorded on a Shimadzu LC-8A (HPLC) PE Sciex API 150EX LCMS. Analytical reversed-phase LCMS analysis was carried out on Luna C18 column (5μ; 30×4.6 mm) under gradient conditions (90% water/0.1% formic acid to 90% acetonitrile/0.1% formic acid) at a flow rate of 4 mL/min. SCX (strong cation exchange) cartridges were purchased from Phenomenex or Biotage.

### Abbreviations

Dimethylformamide (DMF), dichloromethane (DCM), dimethylsulfoxide (DMSO), tetrahydrofuran (THF), high pressure liquid chromatography (HPLC), diisopropylethylamine (DIPEA), triethylamine (TEA), trifluoroacetic acid (TFA), tert-butyloxycarbonyl (Boc), ethylene glycol dimethyl ether (DME), dimethylacetamide (DMA), 1-propanephosphonic acid cyclic anhydride (CycloPhos), 1-hydroxybenzotriazole (HOBt), N-(3-dimethylaminopropyl)-N′-ethylcarbodiimide hydrochloride (EDCl), preparative LCMS refers to preparative high pressure liquid chromatography with mass spectrometric detection.

In the following section, examples of the synthesis of precursors and common intermediates for compounds of the present invention are described.

Amines A-6 were bought from commercial suppliers or were prepared according to the following routes.

### 1-ethyl-6,7-dimethoxy-1,2,3,4-tetrahydroisoquinoline

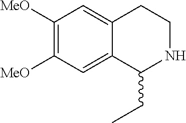

2-(3,4-dimethoxyphenyl)ethanamine (2.76 mmol, 500 mg) was dissolved in DCM (5 ml) and triethylamine (11.04 mmol, 1.551 ml, 1117 mg). Propionic acid (2.76 mmol, 0.206 ml, 204 mg) and then 1-propanephosphonic acid cyclic anhydride (50% wt in ethyl acetate) (4.14 mmol, 2.64 ml) was added. The reaction was stirred at rt for 2 h, then quenched with saturated aq Na₂CO₃ and extracted with DCM. The organic layer was concentrated in vacuo to afford N-(3,4-dimethoxyphenethyl)propionamide (92%, 601 mg, 2.53 mmol) as a brown oil. N-(3,4-dimethoxyphenethyl)propionamide (2.53 mmol, 601 mg) was dissolved in DCM (10 ml). Phosphorous oxychloride (5.07 mmol, 0.472 mL, 777 mg) was added and the reaction mixture was heated in the microwave at 100° C. for 600 s. The reaction was poured portion-wise into a vigorously stirred mixture of saturated aq Na₂CO₃ (50 ml) and DCM (10 ml) over 30 min, the pH was monitored throughout to ensure pH˜10. The aqueous and DCM layers were separated, an additional DCM extraction (1×), and the combined organics were dried over Na₂SO₄ and concentrated in vacuo to afford 1-ethyl-6,7-dimethoxy-3,4-dihydroisoquinoline as a brown oil (92%, 512 mg, 2.335 mmol).

1-ethyl-6,7-dimethoxy-3,4-dihydroisoquinoline (2.289 mmol, 502 mg) was dissolved in MeOH (5 ml), with ice bath cooling, and sodium borohydride (2.289 mmol, 87 mg) was added at 0° C. The reaction was stirred at rt for 30 min. The crude reaction mixture was purified directly by SCX cartridge. The cartridge was washed with methanol to remove non basic bi-products, followed by 2N NH₃ in MeOH washing to elute the product, 1-ethyl-6,7-dimethoxy-1,2,3,4-tetrahydroisoquinoline (87%, 441 mg, 1.993 mmol, >90%). M.S. (ESI) (m/z): 222[M+H]⁺

The following amines were prepared in this fashion:
1-isopropyl-6,7-dimethoxy-1,2,3,4-tetrahydroisoquinoline
1-cyclopropyl-6,7-dimethoxy-1,2,3,4-tetrahydroisoquinoline

### 6-methoxy-2,3,4,5-tetrahydro-1H-benzo[d]azepine

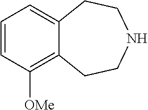

Synthesised according to literature reference WO 2005/082859

### (R)-6,7-dimethoxy-1-methyl-1,2,3,4-tetrahydroisoquinoline

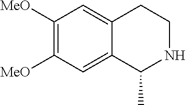

Synthesised according to literature reference, JACS, 1996, 118 (20), p4916-17

### (7-isopropoxy-6-methoxy-1,2,3,4-tetrahydroisoquinolin-1-yl)methanol

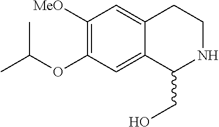

Lithium aluminium hydride (1M in THF) (18.66 ml, 18.66 mmol) was cooled, under an inert atmosphere of argon, in an ice-bath, and 50 ml of dry THF were added. 7-isopropoxy-6-methoxy-1,2,3,4-tetrahydroisoquinoline-1-carboxylic acid (3.3 g, 12.44 mmol), synthesised according to literature reference WO 2009/098283 A1, was then added portionwise over 20 mins. The resulting grey coloured reaction mixture was then allowed to warm up to rt and stirred for 3 h. LCMS analysis indicated complete reaction. The reaction mixture was recooled to 0° C. and then quenched successively with 840 μl of EtOAc, 840 μl of 2M NaOH and 2.5 ml of water. The reaction mixture was then stirred at rt for 2 h and then filtered through celite, washing well with EtOAc. The filtrate was absorbed onto SiO₂ and then purified on a SiO₂ pad, eluting with 5% NH₃ in MeOH in DCM. This yielded (7-isopropoxy-6-methoxy-1,2,3,4-tetrahydroisoquinolin-1-yl)methanol as a beige solid, (1.6 g, 6.37 mmol, 51%). M.S. (ESI) (m/z): 252[M+H]⁺

Central amino acid cores A-2, or alternatively protected derivatives thereof, were bought from commercial suppliers or were prepared according to the following routes.

### (S)-2-(tert-butoxycarbonyl)-1,2,3,4-tetrahydroisoquinoline-3-carboxylic acid

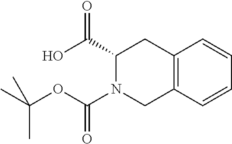

(S)-1,2,3,4-tetrahydroisoquinoline-3-carboxylic acid (3 g, 16.93 mmol) was dissolved in a mixture of dioxane (30 ml) and water (15 ml) and the sodium hydrogen carbonate (2.81 g, 33.9 mmol) was added. Boc₂O (4.06 g, 18.62 mmol) was then added and the reaction mixture was left stirring overnight. The reaction mixture was partitioned between water and DCM. The aqueous layer was extracted twice more with DCM and then the combined organics were washed with water and brine, dried over MgSO₄ and concentrated in vacuo to afford (S)-2-(tert-butoxycarbonyl)-1,2,3,4-tetrahydroisoquinoline-3-carboxylic acid as a colourless gum (4.5 g, 16.23 mmol, 96%).

### (S)-2-(2-fluorobenzyl)-1,2,3,4-tetrahydroisoquinoline-3-carboxylic acid

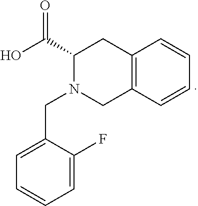

(S)-1,2,3,4-tetrahydroisoquinoline-3-carboxylic acid (2 g, 11.3 mmol) and 2-fluorobenzaldehyde (2.4 ml, 22.57 mmol) were mixed in DMF and AcOH (1.5 ml) was added. The reaction mixture was stirred for 2 h and then the sodium triacetoxyborohydride (4.8 g, 22.57 mmol) was added portionwise and the reaction mixture was left stirring overnight. LCMS analysis indicated complete conversion therefore the reaction mixture was diluted with water (10 ml), MeOH (200 ml) was added and this solution was treated on 2×20 g SCX cartridges, eluting the product with 7M NH₃ in MeOH. This yielded the product as a white solid. This solid was triturated with Et₂O, and then filtered off and dried, yielding (S)-2-(2-fluorobenzyl)-1,2,3,4-tetrahydroisoquinoline-3-carboxylic acid (2 g, 7 mmol, 62%) M.S. (ESI) (m/z): 286[M+H]⁺.

### 5,6,7,8-tetrahydro-1,6-naphthyridine-7-carboxylic acid

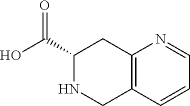

Synthesised according to literature reference, Bioorganic and Medicinal Chemistry 11 (2003) 433-450

### General Method A

### EXAMPLE 1

### (S)-(6,7-dimethoxy-3,4-dihydroisoquinolin-2(1H)-yl)(2-(2-(trifluoromethoxy)benzyl)-1,2,3,4-tetrahydroisoquinolin-3-yl)methanone

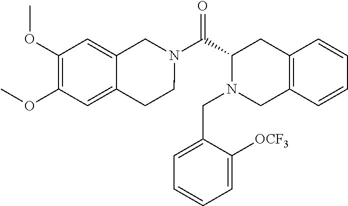

6,7-dimethoxy-1,2,3,4-tetrahydroisoquinoline hydrochloride (2.48 g, 10.82 mmol), (S)-2-(tert-butoxycarbonyl)-1,2,3,4-tetrahydroisoquinoline-3-carboxylic acid (3 g, 10.82 mmol) and triethylamine (3.04 ml, 21.64 mmol) were mixed in DCM (100 ml) and then the Cyclophos (50% in EtOAc) (10.33 g, 16.23 mmol) was added over a few seconds. The reaction mixture was stirred for 1 hr after which time LCMS analysis indicated good conversion. The reaction mixture was quenched with sat. NaHCO₃ (50 ml) and then separated. The organics were washed with water and brine, dried over MgSO₄ and evaporated in vacuo to yield a pale yellow foam. The crude material was then purified on a large SiO₂ pad, eluting with 100% EtOAc, to afford (S)-tert-butyl 3-(6,7-dimethoxy-1,2,3,4-tetrahydroisoquinoline-2-carbonyl)-3,4-dihydroisoquinoline-2(1H)-carboxylate as a white foam (3.8 g, 8.40 mmol, 78%) M.S. (ESI) (m/z): 453[M+H]⁺

(S)-tert-butyl 3-(6,7-dimethoxy-1,2,3,4-tetrahydroisoquinoline-2-carbonyl)-3,4-dihydroisoquinoline-2(1H)-carboxylate (3.8 g, 8.4 mmol) was dissolved in DCM (50 ml) and TFA (5 ml) was added in one portion. The reaction mixture was left stirring overnight. LCMS analysis indicated complete reaction therefore the reaction mixture was evaporated in vacuo, dissolved in MeOH (50 ml) and loaded on a 20 g SCX cartridge, eluting the product with 2M NH₃ in MeOH. This yielded (S)-(6,7-dimethoxy-3,4-dihydroisoquinolin-2(1H)-yl)(1,2,3,4-tetrahydroisoquinolin-3-yl)methanone as a yellow solid (2.5 g, 7.09 mmol, 84%) M.S. (ESI) (m/z): 353[M+H]⁺.

(S)-(6,7-dimethoxy-3,4-dihydroisoquinolin-2(1H)-yl)(1,2,3,4-tetrahydroisoquinolin-3-yl)methanone (30 mg, 0.085 mmol), 2-(trifluoromethoxy)benzaldehyde (65 mg, 0.34 mmol) and sodium triacetoxyborohydride (72 mg, 0.34 mmol) were mixed in DMF (0.6 ml) and heated in the microwave at 100° C. for 5 mins. LCMS analysis indicated good conversion. The reaction mixture was quenched with water (200 μl) and then purified by prep LCMS (XBridge column, 0.1% TFA modifier). Fractions containing the desired product were treated on a 500 mg SCX cartridge eluting with 2M NH₃ in MeOH and then evaporated in vacuo, yielding (S)-(6,7-dimethoxy-3,4-dihydroisoquinolin-2(1H)-yl)(2-(2-(trifluoromethoxy)benzyl)-1,2,3,4-tetrahydroisoquinolin-3-yl)methanone as a white solid (15 mg, 0.028 mmol, 33%) M.S. (ESI) (m/z): 527[M+H]⁺.

The following examples were prepared in this fashion:

### EXAMPLE 2

### (S)-(1-ethyl-6,7-dimethoxy-3,4-dihydroisoquinolin-2(1H)-yl)(2-(2-methoxybenzyl)-1,2,3,4-tetrahydroisoquinolin-3-yl)methanone

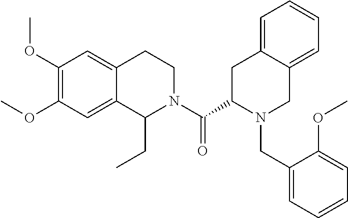

M.S. (ESI) (m/z): 501[M+H]⁺.

### EXAMPLE 3

### (S)-(1-cyclopropyl-6,7-dimethoxy-3,4-dihydroisoquinolin-2(1H)-yl)(2-(2-methoxybenzyl)-1,2,3,4-tetrahydroisoquinolin-3-yl)methanone

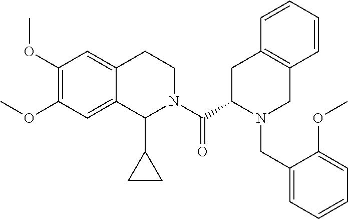

M.S. (ESI) (m/z): 513 [M+H]⁺.

### EXAMPLE 4

### (R)-(6,7-dimethoxy-1-methyl-3,4-dihydroisoquinolin-2(1H)-yl)(6-(2-fluorobenzyl)-5,6,7,13-tetrahydro-1,6-naphthyridin-7-yl)methanone

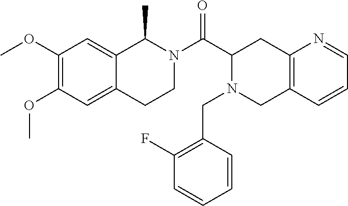

M.S. (ESI) (m/z): 476 [M+H]⁺.

### EXAMPLE 5

### (S)-(1-isopropyl-6,7-dimethoxy-3,4-dihydroisoquinolin-2(1H)-yl)(2-(2-methoxybenzyl)-1,2,3,4-tetrahydroisoquinolin-3-yl)methanone

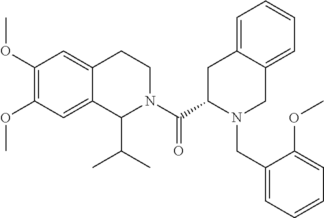

M.S. (ESI) (m/z): 515 [M+H]⁺.

### EXAMPLE 6

### ((S)-2-benzyl-1,2,3,4-tetrahydroisoquinolin-3-yl)((S)-6,7-dimethoxy-1-methyl-3,4-dihydroisoquinolin-2(1H)-yl)methanone

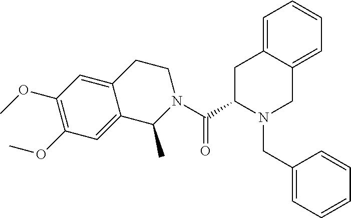

M.S. (ESI) (m/z): 457 [M+H]⁺.

### EXAMPLE 7

### (S)-(2-benzyl-1,2,3,4-tetrahydroisoquinolin-3-yl)(6,7-dimethoxy-3,4-dihydroisoquinolin-2(1H)-yl)methanone

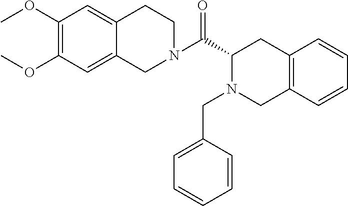

M.S. (ESI) (m/z): 443 [M+H]⁺.

### EXAMPLE 8

### (2-benzyl-5-methyl-1,2,3,4-tetrahydroisoquinolin-3-yl)(6,7-dimethoxy-3,4-dihydroisoquinolin-2(1H)-yl)methanone

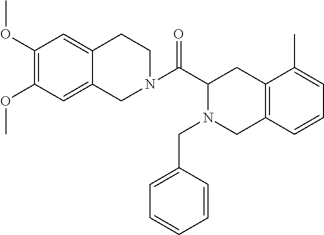

M.S. (ESI) (m/z): 457[M+H]⁺.

### EXAMPLE 9

### (S)-(2-benzyl-1,2,3,4-tetrahydroisoquinolin-3-yl)(6,7-dimethoxy-1-methyl-3,4-dihydroisoquinolin-2(1H)-yl)methanone

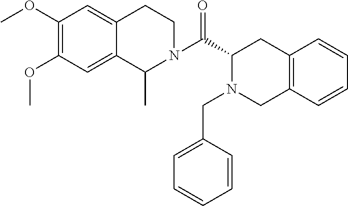

M.S. (ESI) (m/z): 457[M+H]⁺.

### EXAMPLE 10

### (S)-(2-benzyl-1,2,3,4-tetrahydroisoquinolin-3-yl)(6,7-dimethoxy-3-methyl-3,4-dihydroisoquinolin-2(1H)-yl)methanone

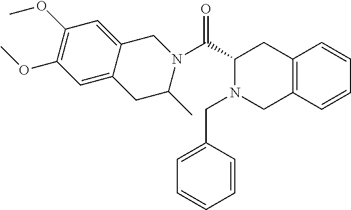

M.S. (ESI) (m/z): 457[M+H]⁺.

### EXAMPLE 11

### (S)-(2-(2-chlorobenzyl)-1,2,3,4-tetrahydroisoquinolin-3-yl)(6,7-dimethoxy-3,4-dihydroisoquinolin-2(1H)-yl)methanone

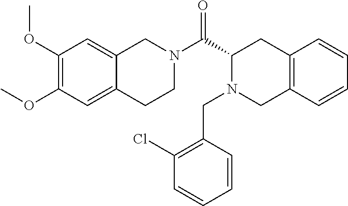

M.S. (ESI) (m/z): 477[M+H]⁺.

### EXAMPLE 12

### (S)-(6,7-dimethoxy-3,4-dihydroisoquinolin-2(1H)-yl)(2-(2-(trifluoromethyl)benzyl)-1,2,3,4-tetrahydroisoquinolin-3-yl)methanone

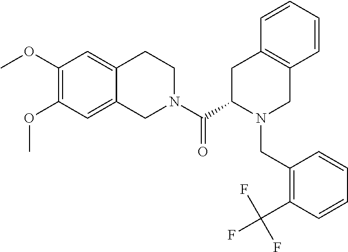

M.S. (ESI) (m/z): 511[M+H]⁺.

### EXAMPLE 13

### (S)-(6,7-dimethoxy-3,4-dihydroisoquinolin-2(1H)-yl)(2-(2-methylbenzyl)-1,2,3,4-tetrahydroisoquinolin-3-yl)methanone

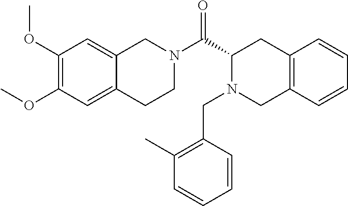

M.S. (ESI) (m/z): 457[M+H]⁺.

### EXAMPLE 14

### (S)-(6,7-dimethoxy-3,4-dihydroisoquinolin-2(1H)-yl)(2-(2-fluorobenzyl)-1,2,3,4-tetrahydroisoquinolin-3-yl)methanone

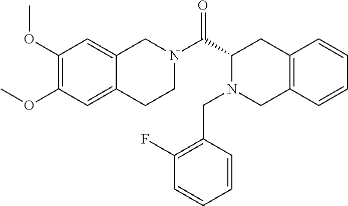

M.S. (ESI) (m/z): 461[M+H]⁺.

### EXAMPLE 15

### (S)-(6,7-dimethoxy-3,4-dihydroisoquinolin-2(1H)-yl)(2-(2-ethoxybenzyl)-1,2,3,4-tetrahydroisoquinolin-3-yl)methanone

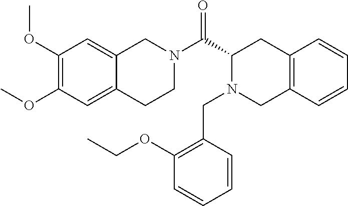

M.S. (ESI) (m/z): 487 [M+H]⁺.

### EXAMPLE 16

### (S)-2-((3-(6,7-dimethoxy-1-methyl-1,2,3,4-tetrahydroisoquinoline-2-carbonyl)-3,4-dihydroisoquinolin-2(1H)-yl)methyl)benzonitrile

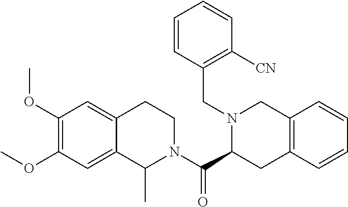

M.S. (ESI) (m/z): 482[M+H]⁺.

### EXAMPLE 17

### (S)-(6,7-dimethoxy-1-methyl-3,4-dihydroisoquinolin-2(1H)-yl)(2-(2-methoxybenzyl)-1,2,3,4-tetrahydroisoquinolin-3-yl)methanone

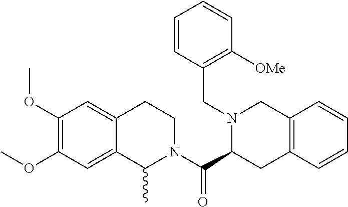

M.S. (ESI) (m/z): 487 [M+H]⁺.

### EXAMPLE 18

### ((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride

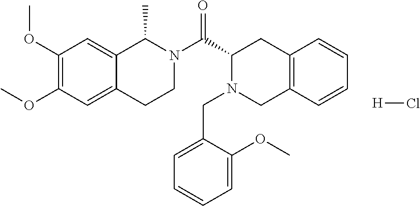

M.S. (ESI) (m/z): 487 [M+H]⁺.

### EXAMPLE 19

### ((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride

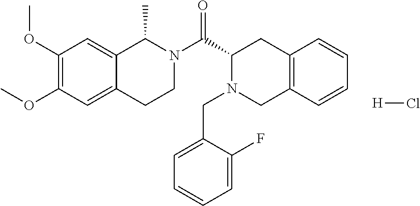

M.S. (ESI) (m/z): 475 [M+H]⁺.

### EXAMPLE 20

### (S)-(6,7-dimethoxy-3-methyl-3,4-dihydroisoquinolin-2(1H)-yl)(2-(2-fluorobenzyl)-1,2,3,4-tetrahydroisoquinolin-3-yl)methanone

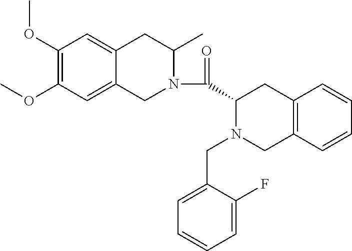

M.S. (ESI) (m/z): 475 [M+H]⁺.

### EXAMPLE 21

### (S)-(6,7-dimethoxy-3-methyl-3,4-dihydroisoquinolin-2(1H)-yl)(2-(2-methoxybenzyl)-1,2,3,4-tetrahydroisoquinolin-3-yl)methanone

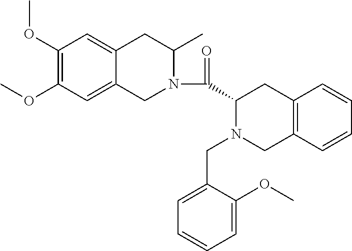

M.S. (ESI) (m/z): 487 [M+H]⁺.

### EXAMPLE 22

### (S)-2-(6,7-dimethoxy-2-(2-(2-methoxybenzyl)-1,2,3,4-tetrahydroisoquinoline-3-carbonyl)-1,2,3,4-tetrahydroisoquinolin-1-yl)acetonitrile

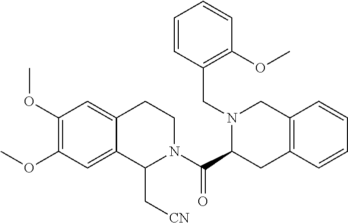

M.S. (ESI) (m/z): 512 [M+H]⁺.

### EXAMPLE 23

### (S)-2-(2-(2-(2-fluorobenzyl)-1,2,3,4-tetrahydroisoquinoline-3-carbonyl)-6,7-dimethoxy-1,2,3,4-tetrahydroisoquinolin-1-yl)acetonitrile

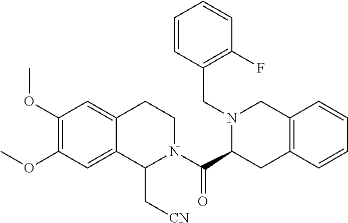

M.S. (ESI) (m/z): 500 [M+H]⁺.

### EXAMPLE 24

### ((R)-6,7-dimethoxy-1-methyl-3,4-dihydroisoquinolin-2(1H)-yl)((S)-2-(2-fluorobenzyl)-1,2,3,4-tetrahydroisoquinolin-3-yl)methanone hydrochloride

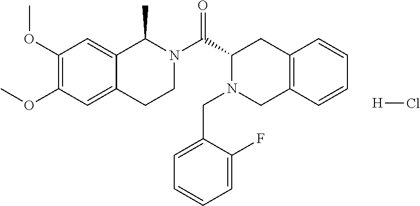

M.S. (ESI) (m/z): 475 [M+H]⁺.

### EXAMPLE 25

### (S)-(6-methoxy-4,5-dihydro-1H-benzo[d]azepin-3(2H)-yl)(2-(2-methoxybenzyl)-1,2,3,4-tetrahydroisoquinolin-3-yl)methanone

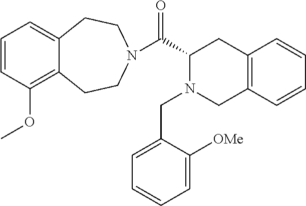

M.S. (ESI) (m/z): 457 [M+H]⁺.

### General Method B-1

### EXAMPLE 26

### (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone

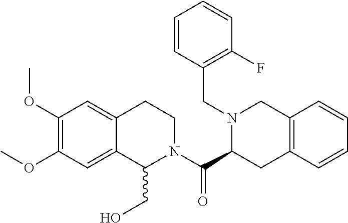

(6,7-dimethoxy-1,2,3,4-tetrahydroisoquinolin-1-yl)methanol (100 mg, 0.448 mmol) and (S)-2-(2-fluorobenzyl)-1,2,3,4-tetrahydroisoquinoline-3-carboxylic acid (128 mg, 0.448 mmol) were mixed in DCM (2 ml) and triethylamine (0.13 ml) was added followed by Cyclophos (50% in EtOAc) (428 mg, 0.672 mmol). The reaction mixture was stirred for 2 h. LCMS analysis indicated good conversion. The reaction mixture was quenched with sat NaHCO₃ (2 ml), diluted with DCM and passed through a hydrophobic frit. The organics were evaporated and then purified by prep HPLC (XBridge column, 0.1% NH₄OH modifier). This yielded (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone (diastereomeric mixture) as a white foam (50 mg, 0.1 mmol, 23%).

The diastereomers were separated by normal phase HPLC (Zorbax RX-SIL column), eluting isocratically with 85% isohexane in EtOAc. First eluted peak (Stereoisomer 1, 13 mg) had ee=94.5% by SFC (Chiralpak AS-H), M.S. (ESI) (m/z): 491.5 [M+H]⁺. and the second eluted peak (Stereoisomer 2, 18 mg) had ee=98.14% by SFC (Chiralpak AS-H), M.S. (ESI) (m/z): 491.5 [M+H]⁺.

### General Method B-2

### EXAMPLE 26

### ((S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone

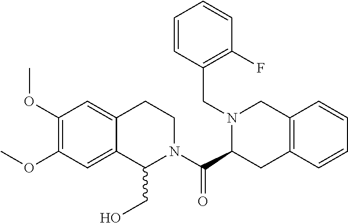

(S)-2-(2-fluorobenzyl)-1,2,3,4-tetrahydroisoquinoline-3-carboxylic acid (1.78 g, 6.24 mmol) and 2,3,4,5,6-pentafluorophenol (1.4 g, 7.49 mmol) were mixed in DCM (60 ml). A crystal of DMAP was added and then the N,N′-diisopropylcarbodiimide (1.46 ml, 9.36 mmol) was added. The reaction was then stirred at rt overnight. TLC (SiO₂, EtOAc) and LCMS analysis showed good conversion. The crude product was concentrated in vacuo and then applied directly to the top of a SiO₂ pad (in a sinter funnel) and the product was eluted with EtOAc:heptane (50:50). This yielded the product as a yellow gum (1.7 g, 3.77 mmol, 60.4%) M.S. (ESI) (m/z): 452[M+H]⁺.

(6,7-dimethoxy-1,2,3,4-tetrahydroisoquinolin-1-yl)methanol (163 mg, 0.731 mmol), (S)-perfluorophenyl 2-(2-fluorobenzyl)-1,2,3,4-tetrahydroisoquinoline-3-carboxylate (330 mg, 0.731 mmol) and diisopropylethylamine (0.12 ml, 0.731 mmol) were mixed in MeCN (3 ml) and heated in the microwave at 120° C. for 20 mins. The reaction mixture was evaporated in vacuo, partitioned between water and DCM and passed through a hydrophobic frit. The crude product was absorbed onto SiO₂ and purified on a 50 g Biotage SNAP cartridge, eluting with 75% EtOAc on heptane, yielding (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone (diastereomeric mixture) as a white foam (300 mg, 0.612 mmol, 84% crude), M.S. (ESI) (m/z): 491.5[M+H]⁺.

### General Method C

### EXAMPLE 26a

### (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 2)

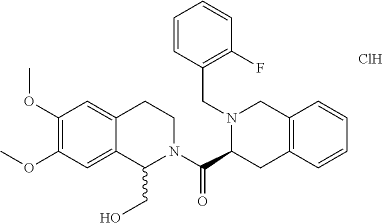

(S)-2-(tert-butoxycarbonyl)-1,2,3,4-tetrahydroisoquinoline-3-carboxylic acid (2 g, 5.77 mmol) and 2,3,4,5,6-pentafluorophenol (1.168 g, 6.35 mmol) were mixed in DCM (20 ml) and cooled to 0° C. A crystal of DMAP was added and then the N,N′-diisopropylcarbodiimide (994 μl, 6.35 mmol) was added. The reaction was then allowed to warm up to rt overnight. TLC (EtOAc:hep 3:7) showed good reaction, product being the first eluted spot. The reaction mixture was absorbed onto SiO₂ and purified on a 90 g Biotage cartridge, eluting with EtOAc:heptane 3:7. This yielded (S)-2-tert-butyl 3-perfluorophenyl 3,4-dihydroisoquinoline-2,3(1H)-dicarboxylate as a colourless gum that solidified on standing (2.2 g, 4.96 mmol, 86%), M.S. (ESI) (m/z): 444[M+H]⁺.

(6,7-dimethoxy-1,2,3,4-tetrahydroisoquinolin-1-yl)methanol (700 mg, 3.14 mmol) and (S)-2-tert-butyl 3-perfluorophenyl 3,4-dihydroisoquinoline-2,3(1H)-dicarboxylate (1.67 g, 3.76 mmol) were mixed in MeCN (12 ml) and heated in the microwave at 120° C. for 10 mins. LCMS analysis indicated complete conversion. The reaction mixture was absorbed onto SiO₂ and purified on a 100 g Biotage SNAP cartridge, eluting with 50%-100% EtOAc in heptane. This yielded (S)-tert-butyl 3-(1-(hydroxymethyl)-6,7-dimethoxy-1,2,3,4-tetrahydroisoquinoline-2-carbonyl)-3,4-dihydroisoquinoline-2(1H)-carboxylate (R_{f} ˜0.4 at 100% EtOAc) as a clear glass (1.1 g, 2.28 mmol, 72%).

(S)-tert-butyl 3-(1-(hydroxymethyl)-6,7-dimethoxy-1,2,3,4-tetrahydroisoquinoline-2-carbonyl)-3,4-dihydroisoquinoline-2(1H)-carboxylate (1.21 g, 2.51 mmol) was dissolved in DCM (15 ml) and TFA (5 ml) was added. The reaction mixture was stirred for 4 h. LCMS showed complete reaction. The reaction mixture was concentrated in vacuo, diluted with MeOH (20 ml) and then treated on a 5 g SCX cartridge, eluting the product with 2M NH₃ in MeOH. This yielded crude (S)-(1-(hydroxymethyl)-6,7-dimethoxy-3,4-dihydroisoquinolin-2(1H)-yl)(1,2,3,4-tetrahydroisoquinolin-3-yl)methanone which was purified by flash column chromatography on a 40 g Biotage SiO₂ cartridge, eluting with 2.5:97.5 2N NH₃ MeOH: DCM increasing to 5:95 2N NH₃ MeOH: DCM to afford in order of elution Stereoisomer 1 (31%, 0.779 mmol, 298 mg) and Stereoisomer 2 (36.7%, 0.92 mmol, 352 mg).

(S)-(1-(hydroxymethyl)-7-isopropoxy-3,4-dihydroisoquinolin-2(1H)-yl)(1,2,3,4-tetrahydroisoquinolin-3-yl)methanone Stereoisomer 1 (100 mg, 0.261 mmol) was dissolved in DMF (2 ml) and 2-fluorobenzaldehyde (83 μl, 0.784 mmol) was added along with 100 μl of acetic acid. The reaction mixture was heated at 100° C. for 5 minutes in the microwave, then sodium triacetoxyborohydride (166 mg, 0.784 mmol) was added and the reaction mixture stirred overnight. LCMS showed good reaction. The reaction mixture was then quenched with water, diluted with MeOH and treated on a 2 g SCX cartridge, eluting basic compounds with 2M NH₃ in MeOH. The crude material was purified by flash column chromatography on silica gel 12 g, eluting with 85:15 EtOAc:isohexane to afford (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone Stereoisomer 2 (59.3%, 0.155 mmol, 76 mg) (ESI) (m/z): 491.5[M+H]⁺.

(S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone Stereoisomer 2 (0.261 mmol, 100 mg) was dissolved in diethyl ether (1 ml) and DCM (10 ml) and excess 2N HCl in diethyl ether was added (˜4 mL). The reaction was concentrated in vacuo to afford a gum which solidified on addition of diethyl ether. The resultant solid was triturated with a mixture of DCM and diethyl ether to afford a white solid, (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 2) (92%, 677 mg, 1.285 mmol) M.S. (ESI) (m/z): 491.5[M+H]⁺.

The following examples were prepared in this fashion:

### EXAMPLE 26b

### (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 1)

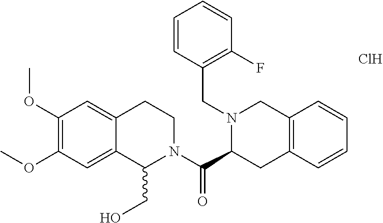

M.S. (ESI) (m/z): 491 [M+H]⁺.

### EXAMPLE 27

### (S)-(6,7-dimethoxy-1-methyl-3,4-dihydroisoquinolin-2(1H)-yl)(2-(2-fluoro-6-methoxybenzyl)-1,2,3,4-tetrahydroisoquinolin-3-yl)methanone

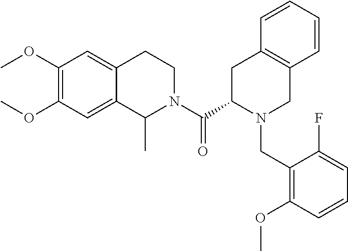

M.S. (ESI) (m/z): 505 [M+H]⁺.

### EXAMPLE 28

### ((R)-6,7-dimethoxy-1-methyl-3,4-dihydroisoquinolin-2(1H)-yl)((S)-2-(3-fluoropyridin-4-yl)methyl)-1,2,3,4-tetrahydroisoquinolin-3-yl)methanone

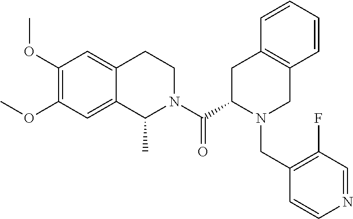

M.S. (ESI) (m/z): 476 [M+H]⁺.

### EXAMPLE 29

### ((R)-6,7-dimethoxy-1-methyl-3,4-dihydroisoquinolin-2(1H)-yl)((S)-2-((2-fluoropyridin-3-yl)methyl)-1,2,3,4-tetrahydroisoquinolin-3-yl)methanone

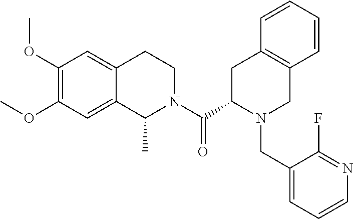

M.S. (ESI) (m/z): 476 [M+H]⁺.

### EXAMPLE 30

### ((R)-6,7-dimethoxy-1-methyl-3,4-dihydroisoquinolin-2(1H)-yl)((S)-2-((2-methoxypyridin-3-yl)methyl)-1,2,3,4-tetrahydroisoquinolin-3-yl)methanone

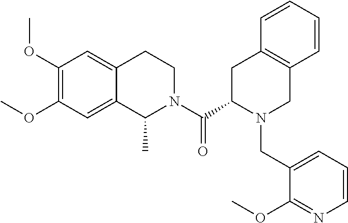

M.S. (ESI) (m/z): 488 [M+H]⁺.

### EXAMPLE 31

### ((R)-6,7-dimethoxy-1-methyl-3,4-dihydroisoquinolin-2(1H)-yl)((S)-2-((3-methoxypyridin-4-yl)methyl)-1,2,3,4-tetrahydroisoquinolin-3-yl)methanone

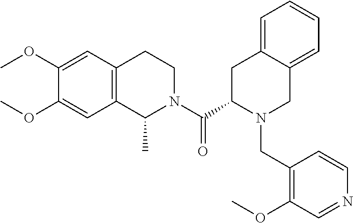

M.S. (ESI) (m/z): 488 [M+H]⁺.

### EXAMPLE 32

### ((R)-6,7-dimethoxy-1-methyl-3,4-dihydroisoquinolin-2(1H)-yl)((S)-2-((4-methoxypyridin-3-yl)methyl)-1,2,3,4-tetrahydroisoquinolin-3-yl)-methanone

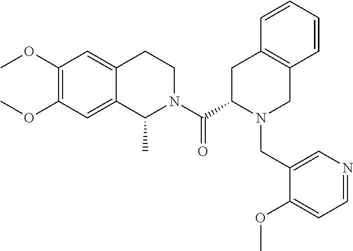

M.S. (ESI) (m/z): 488 [M+H]⁺.

### EXAMPLE 33

### ((R)-6,7-dimethoxy-1-methyl-3,4-dihydroisoquinolin-2(1H)-yl)((S)-2-((4-(trifluoromethyl)pyridin-3-yl)methyl)-1,2,3,4-tetrahydroisoquinolin-3-yl)methanone

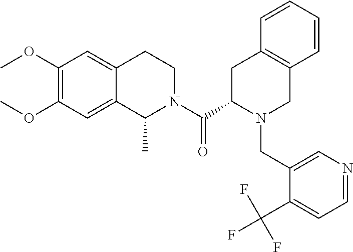

M.S. (ESI) (m/z): 526 [M+H]⁺.

### EXAMPLE 34

### ((S)-2-((3,5-dichloropyridin-4-yl)methyl)-1,2,3,4-tetrahydroisoquinolin-3-yl)((R)-6,7-dimethoxy-1-methyl-3,4-dihydroisoquinolin-2(1H)-yl)methanone

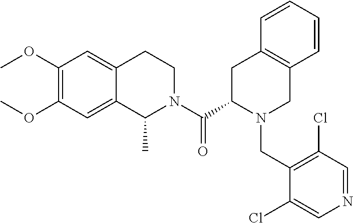

M.S. (ESI) (m/z): 526 [M+H]⁺.

### EXAMPLE 35

### ((S)-2-((1H-indol-7-yl)methyl)-1,2,3,4-tetrahydroisoquinolin-3-yl)((R)-6,7-dimethoxy-1-methyl-3,4-dihydroisoquinolin-2(1H)-yl)methanone

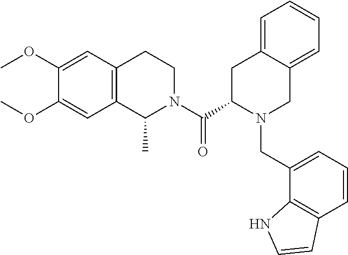

M.S. (ESI) (m/z): 496 [M+H]⁺.

### EXAMPLE 36

### ((S)-2-((6-bromopyridin-2-yl)methyl)-1,2,3,4-tetrahydroisoquinolin-3-yl)((R)-6,7-dimethoxy-1-methyl-3,4-dihydroisoquinolin-2(1H)-yl)methanone

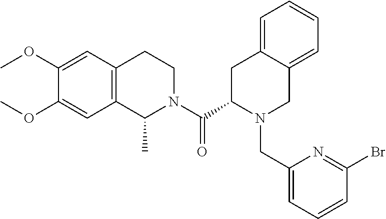

M.S. (ESI) (m/z): 536, 538 [M+H]⁺.

### EXAMPLE 37

### ((S)-2-((1H-indol-4-yl)methyl)-1,2,3,4-tetrahydroisoquinolin-3-yl)((R)-6,7-dimethoxy-1-methyl-3,4-dihydroisoquinolin-2(1H)-yl)methanone

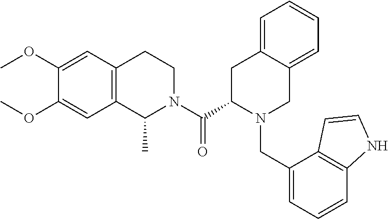

M.S. (ESI) (m/z): 496 [M+H]⁺.

### EXAMPLE 38

### ((S)-2-((1H-indol-6-yl)methyl)-1,2,3,4-tetrahydroisoquinolin-3-yl)((R)-6,7-dimethoxy-1-methyl-3,4-dihydroisoquinolin-2(1H)-yl)methanone

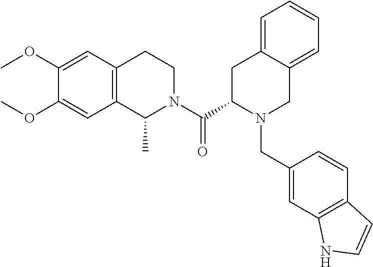

M.S. (ESI) (m/z): 496 [M+H]⁺.

### EXAMPLE 39

### ((S)-2-((3-chloropyridin-4-yl)methyl)-1,2,3,4-tetrahydroisoquinolin-3-yl)((R)-6,7-dimethoxy-1-methyl-3,4-dihydroisoquinolin-2(1H)-yl)methanone

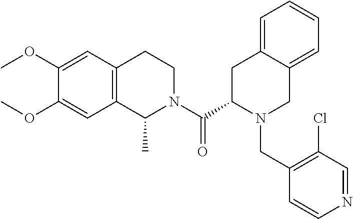

M.S. (ESI) (m/z): 492 [M+H]⁺.

### EXAMPLE 40

### ((R)-6,7-dimethoxy-1-methyl-3,4-dihydroisoquinolin-2(1H)-yl)((S)-2-((4-methylpyridin-3-yl)methyl)-1,2,3,4-tetrahydroisoquinolin-3-yl)methanone

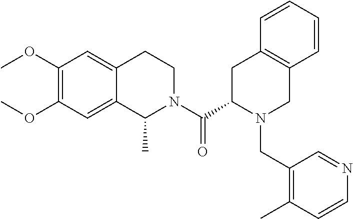

M.S. (ESI) (m/z): 472 [M+H]⁺.

### EXAMPLE 41

### ((R)-6,7-dimethoxy-1-methyl-3,4-dihydroisoquinolin-2(1H)-yl)((S)-2-((3-(trifluoromethyl)pyridin-2-yl)methyl)-1,2,3,4-tetrahydroisoquinolin-3-yl)methanone

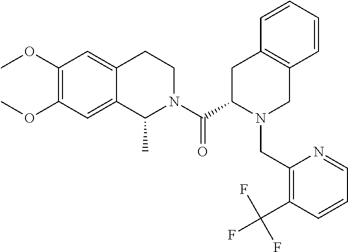

M.S. (ESI) (m/z): 526 [M+H]⁺.

### EXAMPLE 42

### ((S)-2-((4-chloro-1-methyl-1H-pyrazol-3-yl)methyl)-1,2,3,4-tetrahydroisoquinolin-3-yl)((R)-6,7-dimethoxy-1-methyl-3,4-dihydroisoquinolin-2(1H)-yl)methanone

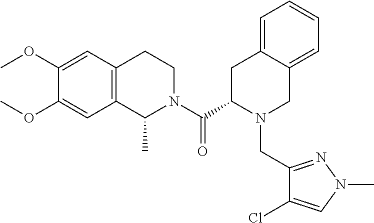

M.S. (ESI) (m/z): 495 [M+H]⁺.

### EXAMPLE 43

### ((R)-6,7-dimethoxy-1-methyl-3,4-dihydroisoquinolin-2(1H)-yl)((S)-2-((3-fluoropyridin-2-yl)methyl)-1,2,3,4-tetrahydroisoquinolin-3-yl)methanone

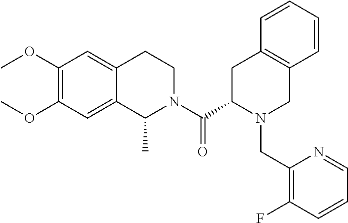

M.S. (ESI) (m/z): 476 [M+H]⁺.

### EXAMPLE 44

### (S)-(6,7-dimethoxy-3,4-dihydroisoquinolin-2(1H)-yl)(2-(2-methoxybenzyl)-1,2,3,4-tetrahydroisoquinolin-3-yl)methanone hydrochloride

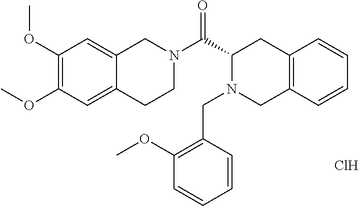

M.S. (ESI) (m/z): 473 [M+H]⁺.

### EXAMPLE 45

### (S)-(2-((4-chloro-1-methyl-1H-pyrazol-3-yl)methyl)-1,2,3,4-tetrahydroisoquinolin-3-yl)(6,7-dimethoxy-3,4-dihydroisoquinolin-2(1H)-yl)methanone hydrochloride

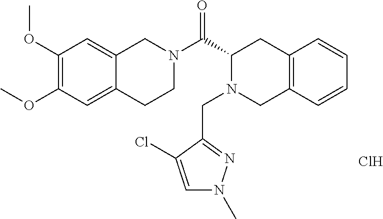

M.S. (ESI) (m/z): 481 [M+H]⁺.

### EXAMPLE 46

### (S)-(2-(2,3-dichlorobenzyl)-1,2,3,4-tetrahydroisoquinolin-3-yl)(6,7-dimethoxy-3,4-dihydroisoquinolin-2(1H)-yl)methanone hydrochloride

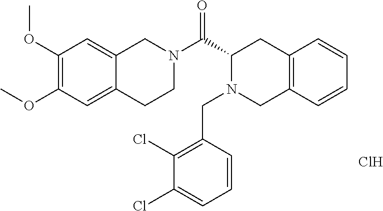

M.S. (ESI) (m/z): 511 [M+H]⁺.

### EXAMPLE 47

### (S)-2-((3-(6,7-dimethoxy-1,2,3,4-tetrahydroisoquinoline-2-carbonyl)-3,4-dihydroisoquinolin-2(1H)-yl)methyl)benzonitrile hydrochloride

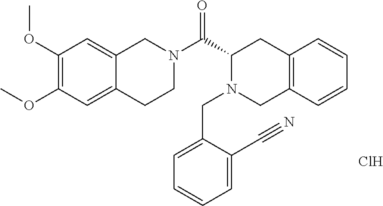

M.S. (ESI) (m/z): 468 [M+H]⁺.

### EXAMPLE 48

### (S)-(2-(2-fluorobenzyl)-1,2,3,4-tetrahydroisoquinolin-3-yl)(1-(hydroxymethyl)-7-isopropoxy-6-methoxy-3,4-dihydroisoquinolin-2(1H)-yl)methanone

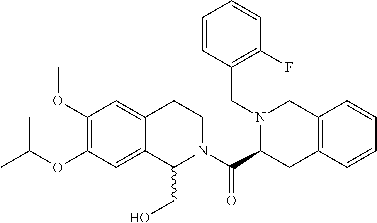

M.S. (ESI) (m/z): 519 [M+H]⁺.

### General Method C-1

### EXAMPLE 49

### (S)-(1-((dimethylamino)methyl)-6,7-dimethoxy-3,4-dihydroisoquinolin-2(1H)-yl)(2-(2-fluorobenzyl)-1,2,3,4-tetrahydroisoquinolin-3-yl)methanone

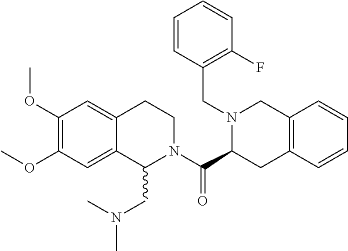

Oxalyl chloride (193 μl, 2.242 mmol) was mixed in DCM (5 ml) and cooled to −60° C. DMSO (319 μl, 4.48 mmol) was then added, followed by (S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone (1 g, 2.04 mmol) in DCM (5 ml). The reaction mixture was stirred for 15 min at −60° C., quenched with TEA (1.43 ml, 10.19 mmol) and then allowed to warm up to rt. The reaction mixture was partitioned between water and DCM and filtered through a hydrophobic frit. LCMS and TLC (100% EtOAc) analysis indicated good conversion. The crude product was absorbed onto SiO₂ and purified on a 50 g Biotage SNAP cartridge, eluting with 25% heptane in EtOAc to afford (S)-2-(2-(2-fluorobenzyl)-1,2,3,4-tetrahydroisoquinoline-3-carbonyl)-6,7-dimethoxy-1,2,3,4-tetrahydroisoquinoline-1-carbaldehyde.

¹H NMR showed a mixture of diastereoisomers and rotamers, aldehyde peaks at 69.4-9.6. M.S. (ESI) (m/z): 489[M+H]⁺.

(S)-2-(2-(2-fluorobenzyl)-1,2,3,4-tetrahydroisoquinoline-3-carbonyl)-6,7-dimethoxy-1,2,3,4-tetrahydroisoquinoline-1-carbaldehyde (80 mg, 0.164 mmol) was dissolved in DCM (3 ml) and dimethylamine hydrochloride (55 mg, 0.655 mmol) was added, along with 50 μl of AcOH. The reaction mixture was left stirring at rt for 1 h, then sodium triacetoxyborohydride was added and the reaction mixture left for a further 2 h. The reaction mixture was quenched with water and the DCM layer separated. The organics were concentrated and the crude product was purified by prep HPLC (XBridge, 0.1% NH₄OH modifier) to afford the two diastereoisomers in order of elution; EXAMPLE 49a: (S)-(1-((dimethylamino)methyl)-6,7-dimethoxy-3,4-dihydroisoquinolin-2(1H)-yl)(2-(2-fluorobenzyl)-1,2,3,4-tetrahydroisoquinolin-3-yl)methanone (Stereoisomer 1) (6.7 mg, 0.013 mmol), M.S. (ESI) (m/z): 518[M+H]⁺; and EXAMPLE 49b: (S)-(1-((dimethylamino)methyl)-6,7-dimethoxy-3,4-dihydroisoquinolin-2(1H)-yl)(2-(2-fluorobenzyl)-1,2,3,4-tetrahydroisoquinolin-3-yl)methanone (Stereoisomer 2) (4 mg, 0.0077 mmol). M.S. (ESI) (m/z): 518[M+H]⁺.

### General Method D

### EXAMPLE 50

### ((R)-6,7-dimethoxy-1-methyl-3,4-dihydroisoquinolin-2(1H)-yl)(2-(2-fluorobenzyl)-1,2,3,4-tetrahydroquinolin-3-yl)methanone

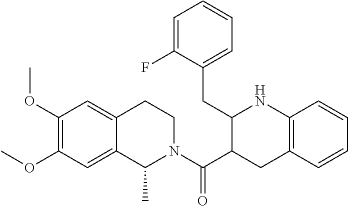

2-chloro-3-quinolinecarboxaldehyde (23.48 mmol, 4.5 g) was suspended in MeCN (100 ml) and a solution of sodium dihydrogen phosphate dihydrate (117 mmol, 94 ml) was added followed by sodium chlorite (70.5 mmol, 6.37 g). The reaction was stirred at rt overnight and then quenched by the addition of sodium sulfite (96 mmol, 96 ml) followed by stirring for 1 h. The aqueous layer was acidified with 2M HCl to pH ˜3 and extracted with EtOAc (2×200 ml). The organic layers were combined, dried over Na₂SO₄ and concentrated in vacuo to afford 2-chloroquinoline-3-carboxylic acid as a pale yellow solid (60.5%, 2.95 g, 14.21 mmol), M.S. (ESI) (m/z): 208[M+H]⁺.

2-chloroquinoline-3-carboxylic acid (4.82 mmol, 1.00 g) and (R)-6,7-dimethoxy-1-methyl-1,2,3,4-tetrahydroisoquinoline (4.82 mmol, 0.99 g) were dissolved in DCM (19.27 ml). N,N′-diisopropylethylamine (19.27 mmol, 3.18 ml, 2.490 g), N-(3-dimethylaminopropyl)-N′ ethylcarbodiimide hydrochloride (5.78 mmol, 1.108 g) and finally 1-hydroxybenzotriazole (5.78 mmol, 0.781 g) were added. The reaction was stirred at rt overnight. The DCM layer was washed with water, saturated citric acid solution and 1N NaOH, dried over Na₂SO₄ and concentrated in vacuo. The crude material was purified by flash column chromatography on silica gel 40 g, with 30:70 EtOAc:heptane increasing to 100% EtOAc to afford (R)-(2-chloroquinolin-3-yl)(6,7-dimethoxy-1-methyl-3,4-dihydroisoquinolin-2(1H)-yl)methanone (80%, 1.53 g, 3.86 mmol).

(R)-(2-chloroquinolin-3-yl)(6,7-dimethoxy-1-methyl-3,4-dihydroisoquinolin-2(1H)-yl)methanone (0.378 mmol, 150 mg) and Pd(PPh₃)₄ (0.038 mmol, 43.7 mg) were dissolved in THF (4 ml) under an atmosphere of N₂ and 2-fluorobenzylzinc chloride 0.5M in THF (0.529 mmol, 1.06 ml) was added. The reaction was heated at 60° C. overnight and then quenched by the addition of water. The crude reaction mixture was extracted with EtOAc, dried over Na₂SO₄ and concentrated in vacuo. The crude material was purified by flash column chromatography on silica gel, eluting with 30:70 EtOAc:heptane to afford (R)-(6,7-dimethoxy-1-methyl-3,4-dihydroisoquinolin-2(1H)-yl)(2-(2-fluorobenzyl)quinolin-3-yl)methanone (93%, 166 mg, 0.353 mmol). M.S. (ESI) (m/z): 471[M+H]⁺.

(R)-(6,7-dimethoxy-1-methyl-3,4-dihydroisoquinolin-2(1H)-yl)(2-(2-fluorobenzyl)quinolin-3-yl)methanone (0.117 mmol, 55 mg) was dissolved in AcOH (1 ml) and platinum(IV)oxide hydrate (0.020 mmol, 5 mg) was added. The reaction was stirred at rt under an atmosphere of 3 bar H₂(g) overnight. The reaction mixture was filtered through a pad of dicalite, washing with MeOH and solvents removed in vacuo to afford the crude material. The crude product was purified by prep HPLC (C18 Xbridge, with 0.1% NH₄OH in MeCN/water as modifier) to afford ((R)-6,7-dimethoxy-1-methyl-3,4-dihydroisoquinolin-2(1H)-yl)(2-(2-fluorobenzyl)-1,2,3,4-tetrahydroquinolin-3-yl)methanone stereoisomer 1_(5%, 2.6 mg, 0.0055 mmol) and stereoisomer 2 (3%, 1.7 mg, 0.0035 mmol). M.S. (ESI) (m/z): 475 [M+H]⁺for both isomers.

### OX1R Orexin Receptor Radioligand Binding Assays.

OX1R orexin receptor binding assays were performed in 25 mM HEPES, 0.5 mM EDTA, 2.5 mM MgCl₂, 0.3% BSA. The test compounds were solubilised in DMSO to make stock solutions of 10⁻²M, and assayed over a 10 point half-log concentration range starting at 10⁻⁵M. Briefly, homogenates from CHO cells stably expressing the human OX1R orexin receptor were incubated with 8 nM [³H] SB674042 in the presence of increasing concentrations of test or reference compounds. Following incubation at room temperature for 2.5 hours the assay was terminated by filtering through GF/B filters, which were then washed with three volumes of assay buffer (25 mM HEPES, 0.5 mM EDTA, 2.5 nM MgCl2) containing 0.01% BSA. The amount of radiolabel remaining on the filters was then determined by scintillation counting and used to determine percentage effect relative to the OX1R receptor antagonist SB674042 (1 μM). Data were analysed by non-linear regression to calculate affinities of the compounds for the receptor. Results were calculated from two independent observations, performed in duplicate with a single analysis of the combined data being performed.

### OX2R Orexin Receptor Radioligand Binding Assays.

OX2R orexin receptor binding assays were performed in 25 mM HEPES, 0.5 mM EDTA, 2.5 mM MgCl₂, 0.3% BSA. The test compounds were solubilised in DMSO to make stock solutions of 10⁻²M, and assayed over a 10 point half-log concentration range starting at 10⁻⁵M. Briefly, homogenates from CHO cells stably expressing the human OX2R orexin receptor were incubated with 45 nM [³H] (S)-1-(6,7-dimethoxy-3,4-dihydroisoquinolin-2(1H)-yl)-3,3-dimethyl-2-(pyridin-4-ylmethylamino)butan-1-one (Compound 29 Hirose M. et al, Bioorganic and Medicinal Chemistry Letters, 2003, 13:4497-4499) in the presence of increasing concentrations of test or reference compounds. Following incubation at room temperature for 2.5 hours the assay was terminated by filtering through GF/B filters, which were then washed with three volumes of assay buffer (25 mM HEPES, 0.5 mM EDTA, 2.5 nM MgCl₂) containing 0.01% BSA. The amount of radiolabel remaining on the filters was then determined by scintillation counting and used to determine percentage effect relative to the OX2R receptor antagonist JNJ-10397049 1-(2,4-dibromophenyl)-3-((4S,5S)-2,2-dimethyl-4-phenyl-1,3-dioxan-5-yl)urea (1 μM). Data were analysed by non-linear regression to calculate affinities of the compounds for the receptor. Results were calculated from two independent observations, performed in duplicate with a single analysis of the combined data being performed.

### OX1R Orexin Receptor Functional Assays.

The functional activity of compounds at the OX1R receptor was determined by measuring the effect on the intracellular increase in calcium elicited by the agonist orexin A. CHO. K1 cells stably expressing the human OX1R were seeded at 15,000 cells/well. Functional assays were performed 1×PBS containing 1×fluo 4-nw (Invitrogen), 2 nM probenicid. The test compounds were solubilised in DMSO to make stock solutions of 10⁻²M, and assayed over a 10 point half-log concentration range starting at 10⁻⁵M. Briefly, cells were incubated in the presence of increasing concentrations of test or reference compounds. Following incubation at room temperature for 20 minutes the agonist orexin A was added (final concentration 0.75 nM) and the increase in intracellular calcium was determined using a fluorescent plate reader. The agonist response was determined from the fluorescence (λₑₓ=488 nm, λₑₘ=540 nm) and used to determine percentage effect of compounds relative to the OX1R receptor antagonist SB674042 (10 μM). Data were analysed by non-linear regression to calculate activity of the compounds for the receptor. Results were calculated from two independent observations, performed in duplicate with a single analysis of the combined data being performed.

### OX2R Orexin Receptor Functional Assays.

The functional activity of compounds at the OX2R receptor was determined by measuring the effect on the intracellular increase in calcium elicited by the agonist orexin A. CHO.K1 cells stably expressing the human OX2R were seeded at 15,000 cells/well. Functional assays were performed 1×PBS containing 1×fluo 4-nw (Invitrogen), 2 nM probenicid. The test compounds were solubilised in DMSO to make stock solutions of 10⁻²M, and assayed over a 10 point half-log concentration range starting at 10⁻⁵M. Briefly, cells were incubated in the presence of increasing concentrations of test or reference compounds. Following incubation at room temperature for 20 minutes the agonist orexin A was added (final concentration 0.75 nM) and the increase in intracellular calcium was determined using a fluorescent plate reader. The agonist response was determined from the fluorescence (λₑₓ=488 nm, λₑₘ=540 nm) and used to determine percentage effect of compounds relative to the OX2R receptor antagonist JNJ-10397049 1-(2,4-dibromophenyl)-3-((4S,5S)-2,2-dimethyl-4-phenyl-1,3-dioxan-5-yl)urea (10 μM). Data were analysed by non-linear regression to calculate activity of the compounds for the receptor. Results were calculated from two independent observations, performed in duplicate with a single analysis of the combined data being performed.

In particular, compounds of the present invention had activity in antagonising the orexin-2 receptor in the aforementioned assays, generally with an IC₅₀ of less than 10 μM. Many of the compounds in the present invention had activity antagonising the orexin-2 receptor with an IC₅₀ of less than 100 nM.

1-15. (canceled)

16. A heterocyclic derivative having the formula I 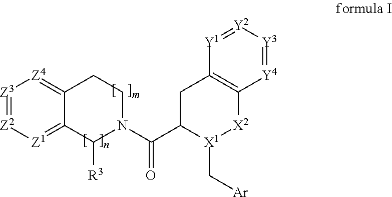
wherein
X¹ and X² are independently CH or N with the proviso that one of X¹ and X² is N;
Y¹-Y⁴ are CR¹ or 1-2 of Y¹ to Y⁴ are N;
Z¹-Z⁴ are CR² or 1-2 of Z¹ to Z⁴ are N;
Each R¹ and R² is independently H, C₁₋₆alkyl, C₂₋₆alkenyl, C₂₋₆alkynyl, C₁₋₆alkoxy, C₂₋₆alkenyloxy, C₃₋₇cycloalkyl, C₃₋₇cycloalkyloxy, OH, halogen or CN said C₁₋₆alkyl and C₁₋₆alkoxy being optionally substituted with one or more halogens;
Each R³ is independently H, C₁₋₆alkyl or C₃₋₇cycloalkyl said C₁₋₆alkyl and C₃₋₇cycloalkyl being optionally substituted with one or more substituent independently selected from halogen, OH, CN, NR⁴R⁵ and C₁₋₆alkoxy;
R⁴ and R⁵ are independently H or C₁₋₆alkyl or R⁴ and R⁵ together with the N to which they are bonded form a 4-7 membered heterocyclic ring;
Ar is C₆₋₁₀aryl or a 5-10 membered heteroaryl ring system comprising 1-3 heteroatoms independently selected from N, O and S, said C₆₋₁₀aryl or 5-10 membered heteroaryl being optionally substituted with 1-3 R⁶;
Each R⁶ is independently selected from C₁₋₆alkyl, C₂₋₆alkenyl, C₂₋₆alkynyl, C₁₋₆alkoxy, C₂₋₆alkenyloxy, C₃₋₇cycloalkyl, C₃₋₇cycloalkyloxy, OH, halogen, CN and a 5-6 membered heteroaryl comprising 1-2 heteroatoms selected from N, O and S, said C₁₋₆alkyl and C₁₋₆alkoxy being optionally substituted with one or more halogens;
m is 0 or 1 and
n is 1 or 2
or a pharmaceutically acceptable salt thereof.

17. The heterocyclic derivative according to claim 16 or a pharmaceutically acceptable salt thereof, wherein X¹ is N and X² is CH.

18. The heterocyclic derivative according to claim 16 or a pharmaceutically acceptable salt thereof, wherein Y¹-Y⁴ are CR¹ and each R¹ is independently H or methyl

19. The heterocyclic derivative according to claim 16 or a pharmaceutically acceptable salt thereof, wherein one or two of Z¹-Z⁴ are C(OCH₃) and the others are CH.

20. The heterocyclic derivative according to claim 19 or a pharmaceutically acceptable salt thereof, wherein Z¹ and Z⁴ are CH and Z² and Z³ are C(OCH₃).

21. The heterocyclic derivative according to claim 16 or a pharmaceutically acceptable salt thereof, wherein R³ is H, methyl, CH₂OH, or CH₂CN.

22. The heterocyclic derivative according to claim 16 or a pharmaceutically acceptable salt thereof, wherein Ar is phenyl optionally substituted with 1-2 substituents selected from fluoro, chloro, methyl, trifluoromethyl, methoxyl, trifluoromethoxyl, ethoxyl, and CN.

23. The heterocyclic derivative according to claim 16 or a pharmaceutically acceptable salt thereof, wherein Ar is pyridyl optionally substituted with 1-2 substituents selected from fluoro, chloro, methyl, trifluoromethyl, methoxyl, trifluoromethoxyl, ethoxyl, and CN.

24. The heterocyclic derivative according to claim 16 or a pharmaceutically acceptable salt thereof, wherein m and n are both 1.

25. A heterocyclic derivative according to claim 16 which is selected from:
(S)-(6,7-dimethoxy-3,4-dihydroisoquinolin-2(1H)-yl)(2-(2-(trifluoromethoxy)benzyl)-1,2,3,4-tetrahydroisoquinolin-3-yl)methanone;
(S)-(1-ethyl-6,7-dimethoxy-3,4-dihydroisoquinolin-2(1H)-yl)(2-(2-methoxybenzyl)-1,2,3,4-tetrahydroisoquinolin-3-yl)methanone;
(S)-(1-cyclopropyl-6,7-dimethoxy-3,4-dihydroisoquinolin-2(1H)-yl)(2-(2-methoxybenzyl)-1,2,3,4-tetrahydroisoquinolin-3-yl)methanone;
(S)-(2-benzyl-1,2,3,4-tetrahydroisoquinolin-3-yl)(6,7-dimethoxy-3,4-dihydroisoquinolin-2(1H)-yl)methanone;
(S)-(2-benzyl-1,2,3,4-tetrahydroisoquinolin-3-yl)(6,7-dimethoxy-1-methyl-3,4-dihydroisoquinolin-2(1H)-yl)methanone;
(S)-(2-(2-chlorobenzyl)-1,2,3,4-tetrahydroisoquinolin-3-yl)(6,7-dimethoxy-3,4-dihydroisoquinolin-2(1H)-yl)methanone;
(S)-(6,7-dimethoxy-3,4-dihydroisoquinolin-2(1H)-yl)(2-(2-(trifluoromethyl)benzyl)-1,2,3,4-tetrahydroisoquinolin-3-yl)methanone;
(S)-(6,7-dimethoxy-3,4-dihydroisoquinolin-2(1H)-yl)(2-(2-fluorobenzyl)-1,2,3,4-tetrahydroisoquinolin-3-yl)methanone;
(S)-(6,7-dimethoxy-1-methyl-3,4-dihydroisoquinolin-2(1H)-yl)(2-(2-methoxybenzyl)-1,2,3,4-tetrahydroisoquinolin-3-yl)methanone;
((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride;
((S)-6,7-dimethoxy-1-methyl-3,4-dihydroisoquinolin-2(1H)-yl)((S)-2-(2-methoxybenzyl)-1,2,3,4-tetrahydroisoquinolin-3-yl)methanone hydrochloride;
(S)-2-(6,7-dimethoxy-2-(2-(2-methoxybenzyl)-1,2,3,4-tetrahydroisoquinoline-3-carbonyl)-1,2,3,4-tetrahydroisoquinolin-1-yl)acetonitrile;
(S)-2-(2-(2-(2-fluorobenzyl)-1,2,3,4-tetrahydroisoquinoline-3-carbonyl)-6,7-dimethoxy-1,2,3,4-tetrahydroisoquinolin-1-yl)acetonitrile;
((R)-6,7-dimethoxy-1-methyl-3,4-dihydroisoquinolin-2(1H)-yl)((S)-2-(2-fluorobenzyl)-1,2,3,4-tetrahydroisoquinolin-3-yl)methanone hydrochloride;
(S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 2);
(S)-(2-(2-fluorobenzyl)-1,2,3,4-tetrahydroisoquinolin-3-yl)(1-(hydroxymethyl)-6,7-dimethoxy-3,4-dihydroisoquinolin-2(1H)-yl)methanone hydrochloride (Stereoisomer 1);
(S)-(6,7-dimethoxy-1-methyl-3,4-dihydroisoquinolin-2(1H)-yl)(2-(2-fluoro-6-methoxybenzyl)-1,2,3,4-tetrahydroisoquinolin-3-yl)methanone;
((R)-6,7-dimethoxy-1-methyl-3,4-dihydroisoquinolin-2(1H)-yl)((S)-2-((4-methoxypyridin-3-yl)methyl)-1,2,3,4-tetrahydroisoquinolin-3-yl)methanone;
((R)-6,7-dimethoxy-1-methyl-3,4-dihydroisoquinolin-2(1H)-yl)((S)-2-((4-(trifluoromethyl)pyridin-3-yl)methyl)-1,2,3,4-tetrahydroisoquinolin-3-yl)methanone;
((S)-2-((1H-indol-4-yl)methyl)-1,2,3,4-tetrahydroisoquinolin-3-yl)((R)-6,7-dimethoxy-1-methyl-3,4-dihydroisoquinolin-2(1H)-yl)methanone;
((S)-2-((3-chloropyridin-4-yl)methyl)-1,2,3,4-tetrahydroisoquinolin-3-yl)((R)-6,7-dimethoxy-1-methyl-3,4-dihydroisoquinolin-2(1H)-yl)methanone;
((R)-6,7-dimethoxy-1-methyl-3,4-dihydroisoquinolin-2(1H)-yl)((S)-2-((3-(trifluoromethyl)pyridin-2-yl)methyl)-1,2,3,4-tetrahydroisoquinolin-3-yl)methanone;
((S)-2-((4-chloro-1-methyl-1H-pyrazol-3-yl)methyl)-1,2,3,4-tetrahydroisoquinolin-3-yl)((R)-6,7-dimethoxy-1-methyl-3,4-dihydroisoquinolin-2(1H)-yl)methanone ;
((R)-6,7-dimethoxy-1-methyl-3,4-dihydroisoquinolin-2(1H)-yl)((S)-2-((3-fluoropyridin-2-yl)methyl)-1,2,3,4-tetrahydroisoquinolin-3-yl)methanone;
(S)-(6,7-dimethoxy-3,4-dihydroisoquinolin-2(1H)-yl)(2-(2-methoxybenzyl)-1,2,3,4-tetrahydroisoquinolin-3-yl)methanone hydrochloride;
(S)-(2-(2,3-dichlorobenzyl)-1,2,3,4-tetrahydroisoquinolin-3-yl)(6,7-dimethoxy-3,4-dihydroisoquinolin-2(1H)-yl)methanone hydrochloride and
(S)-(2-(2-fluorobenzyl)-1,2,3,4-tetrahydroisoquinolin-3-yl)(1-(hydroxymethyl)-7-isopropoxy-6-methoxy-3,4-dihydroisoquinolin-2(1H)-yl)methanone
or a pharmaceutically acceptable salt thereof.

26. A method of treating a neurological or psychiatric disorder or disease in which orexin receptors are involved, the method comprising administering to a subject in need thereof a therapeutically effective amount of a heterocyclic derivative according to claim 16 or a pharmaceutically acceptable salt thereof.

27. The method according to claim 26, wherein the neurological disorder or disease is a sleep disorder.

28. The method according to claim 26, wherein the disease is obesity or diabetes.

29. A pharmaceutical composition comprising a heterocyclic derivative according to claim 16 or a pharmaceutically acceptable salt thereof in admixture with one or more pharmaceutically acceptable excipients.
